(19) United States
(12) Patent Application Publication
(10) Pub. No.: US 2019/0376030 A1
(43) Pub. Date: Dec. 12, 2019

(54) REGULATORY T CELL POPULATIONS

(71) Applicant: Memorial Sloan Kettering Cancer Center, New York, NY (US)

(72) Inventors: Alexander Y. Rudensky, New York, NY (US); Andrew G. Levine, Livingston, NJ (US)

(21) Appl. No.: 16/433,251

(22) Filed: Jun. 6, 2019

Related U.S. Application Data
(60) Provisional application No. 62/681,570, filed on Jun. 6, 2018.

(51) Int. Cl. C12N 5/0783; C07K 14/47; A61K 35/17

(52) U.S. Cl. CPC C12N 5/0637; C07K 14/47; C12N 2501/60; C12N 2501/2327; C12N 2501/24; A61K 35/17

(57) ABSTRACT

The present disclosure provides regulatory T cells and regulatory T cell populations engineered to express a transcription factor. The present disclosure provides for treatment of immune disorders with regulatory T cells and regulatory T cell populations engineered to express a transcription factor.

### GOVERNMENT SUPPORT

This invention was made with government support under R37AI034206 awarded by the National Institutes of Health. The government has certain rights in the invention.

### BACKGROUND

With advancements in understanding of immune systems additional avenues for therapeutics arise. There is a need to identify novel compositions and methods of treatment to treat disease using the immune system.

### SUMMARY

The present disclosure encompasses the recognition that novel therapies can be developed to treat diseases, disorders, or conditions through the engineering of cells of the immune system. In some embodiments, the present disclosure recognizes that some diseases, disorders, or conditions, e.g. inflammatory and autoimmune diseases, can be a result of an overactive and or self-reactive immune system. In some embodiments, the present disclosure recognizes regulatory T-cells (Treg) can be a useful tool to regulate an overactive and or self-reactive immune system. In some embodiments, the present disclosure relates to engineering Treg cells to treat diseases, disorders, or conditions, e.g. inflammatory and autoimmune diseases. In some embodiments, the present disclosure recognizes that engineering a Treg cell to express a transcription factor of interest can provide a novel therapeutic for the treatment of inflammatory and autoimmune diseases. In some embodiments, the present disclosure recognizes that engineering a Treg cell to express transcription factor Tbet can provide a novel therapeutic for the treatment of inflammatory and autoimmune diseases.

In some embodiments, the present disclosure provides an isolated population of regulatory T (Treg) cells which have been engineered to express Tbet. In some embodiments, the present disclosure provides an isolated population of regulatory T (Treg) cells characterized by an ability to suppress an immune response when contacted with a system undergoing or at risk of the immune response. In some embodiments, the present disclosure provides a method of suppressing a T_{H}1 type immune response the method comprising administering to a subject a population of Tregs which have been engineered to express Tbet. In some embodiments, the present disclosure provides a method of preparing a specialized Treg population, the method comprising: obtaining an initial Treg cell or population; culturing the initial Treg cell or population for a period of time and under conditions sufficient that a specialized Treg population characterized in that Tbet expression is increased 2, 5, 10, 20, 30, 40, 50, 60 fold relative to a reference is prepared.

### BRIEF DESCRIPTION OF THE DRAWING

FIGS. 1a-1d demonstrate stable T-bet expression in a subset of peripheral Treg cells. 1a, Splenic cells in Tbx21RFP-creERT2 mice 3 weeks after tamoxifen gavage on days −2 and 0. Numbers on graph indicate the mean. Data are mean±s.e.m. 1b, Schematic of tamoxifen administration to Tbx21RFP-creERT2 mice (top) and flow cytometry (bottom) of splenic CD4 Thy1.1+ and Thy1.1− cells. 1c, Upper, RFP+(left axis, squares) and YFP+(right axis, circles) Treg cells. Lower, percentage of RFP+ of YFP+ Treg cells 3 weeks (white symbols), 3 months (grey symbols), and 7 months (black symbols) after tamoxifen gavage. 1d, Upper, schematic of tamoxifen treatment and N. brasiliensis infection. Lower, percentage of RFP+ among YFP+ Treg cells in mice challenged with PBS (white circles) and N. brasiliensis (Nb; black circles); (bottom, right) RFP expression in Treg (shaded histograms) or YFP+ Treg (open histograms) cells from spleens of mice challenged with PBS (black) or N. brasiliensis (red). Data are mean. Two-tailed t-test (NS, not significant). All data are representative of 2 experiments, n≥3 mice per group each.

FIGS. 2a-2f demonstrate stable differentiation of T-bet+ Treg cells in response to L. monocytogenes infection. 2a, Schematic of experiment shown in b combining tamoxifen (TX) treatment and L. monocytogenes (Lm) infection in Tbx21RFP-creERT2 mice. 2b, Percentage of RFP+, YFP+, and YFP+/RFP+ ratio in CD4+ Thy1.1+(left) and Thy1.1− (right) cells in spleens and livers of mice challenged with PBS and L. monocytogenes. 2c, Schematic of experiments shown in d, e and f; 1° and 2° indicate primary and secondary challenge, respectively. 2d, Data presented as in b. 2e, Percentage of RFP+ of YFP+ Treg cells. 2f, Data presented as in b. Bars, mean. Two-tailed t-test (***P<0.001, **P<0.01, and *P<0.05, respectively; NS, not significant). All data are representative of ≥2 experiments, n≥4 mice per group each.

FIGS. 3a-3k demonstrate Foxp3 ablation in T-bet+ Treg cells results in spontaneous TH1 autoimmune disease. 3a, Body weights of 8-10-week-old Tbx21RFP-creFoxp3WT (grey circles), Tbx21RFP-creFoxp3fl (red circles), Tbx21RFP-cre Foxp3WT/WT (blue circles), and Tbx21RFP-creFoxp3fl/WT (white circles) mice. 3b, Haematoxylin and eosin staining (left) and histology scores (right) of lungs from Tbx21RFP-cre mice combined with indicated Foxp3 alleles, treated or not with antibiotics (ABX). Tbx21RFP-creFoxp3fl mice show moderate perivascular and peribronchiolar inflammation, mild respiratory epithelial hyperplasia and mucus metaplasia with hyalinization (arrows). Pulmonary arterioles are contracted with thickened media, reactive endothelia and marginating leukocytes (arrowheads). Original magnification, 20×. 3c, 3d, Lymph node cell numbers (3c) and characterization of T cell populations in spleens (3d). 3e, Flow cytometry of splenic cells in Tbx21RFP-creFoxp3WT (left) and Tbx21RFP-creFoxp3fl (right) mice, gated on fixed CD4+(top) and live CD4+CD25− (bottom) cells. 3f, Quantification of RFP− and RFP+ CD4 T cells, as shown in e (bottom). 3g, RFP expression (left) and cytokine production (right) in splenic CD8 T cells. 3h, Cytokine production by splenic CD4+Foxp3− T cells. 3i, Representative images (left) and insets (right) of spleen sections from Tbx21RFP-cre mice with CD4 (green, top) or CD8 (green, bottom), RFP (red), Foxp3 (blue) and CD44 (grey). Inset, arrowheads indicate CD4+CD44hiRFP+Foxp3−(top) or CD8+CD44hiRFP+(bottom) cells and arrows indicate CD4+CD44hiRFP+Foxp3+ cells. 3j, 3k, Nearest distances between cells as shown in i. Foxp3+ denotes CD4+CD44hiFoxp3+; Foxp3− (3j) denotes CD4+CD44hiFoxp3− and CD8+(3k) denotes CD8+CD44hiRFP+. Each circle (3j, 3k) represents the distance between cells on imaged sections from three mice. Data are mean±s.e.m. Two-tailed t-test (***P<0.001, **P<0.01; NS, not significant). All data are representative of several experiments.

FIGS. 4a-4e demonstrate acute ablation of T-bet+ Treg cells results in TH1 immune activation. Bone marrow chimaeric mice were injected with 0.5 μg diphtheria toxin (DT) on day 0, then treated daily with 0.1 μg of diphtheria toxin until day 15. 4a, Weight loss in the indicated mice. 4b, Flow cytometry of splenic CD4 (top) and Treg (bottom) cells in the indicated mice. 4c, Activation status of CD45.1+ and CD45.2+ Treg cell compartments in spleens of indicated mice. 4d, 4e, T cell activation (4d) and cytokine production (4e) in control (white circles) and T-bet-depleted (black circles) chimaeras. Data are mean±s.e.m. Two-tailed t-test (**P<0.01, *P<0.05; NS, not significant). Data are representative of 2 experiments, n≥6 mice per group.

FIGS. 5a-5f demonstrates T-bet T_{reg} cells suppress T_{H}1 and CD8₊ T cells, but not T_{H}2 or T_{H}17 responses. 5a, Schematic for tamoxifen administration and depletion of non-T-bet-expressing T_{reg} cells in Foxp3_{fl-DTR} Tbx21_{RFP-creERT2} mice. 5b, Flow cytometry of splenic CD4 T cells in the indicated mice on day 9, as outlined in 5a. 5c-5f, T_{reg} cell percentages (5c), and activated (5d) and cytokine-producing (5e, 5f) T cells in spleens of tamoxifen-treated Foxp3_{Thy1.1}Tbx21_{RFP-creERT2} (open circles), oil-treated Foxp3_{fl-DTR} Tbx21_{RFP-creERT2} mice (black circles) and tamoxifen-treated Foxp3_{fl-DTR} Tbx21_{RFP-creERT2} (grey circles) mice. Data are mean±s.e.m. Two-tailed t-test (***P<0.001, **P<0.01, *P<0.05; NS, not significant). Data are representative of 2 experiments, n≥2 mice per group each.

FIGS. 6a-6i show analysis of T-bet+ cells in Tbx21RFP-creERT2 reporter mice. 6a, Targeting strategy for the Tbx21 locus. 6b, T-bet protein levels in immune cells in Tbx21RFP-creERT2 mice. 6c, T-bet protein levels in Tbx21RFP-creERT2 mice gavaged with tamoxifen on days −2 and 0 and analysed 3 weeks later. Shaded grey and open histograms represent all and YFP+ cells, respectively. 6d, Flow cytometry of RFP expression in Treg and non-Treg CD4 T cells. 6e, Flow cytometry of splenic Treg cells. 6f, Percentage of CD44hiCD62Llo among Thy1.1+(top) and RFP+ among CD44hiCD62LloThy1.1+(bottom) cells in Tbx21RFP-creERT2 3 weeks (white squares), 3 months (grey squares), and 7 months (black squares) after tamoxifen treatment. 6g, Flow cytometry of T-bet expression in GATA3+(blue gate, left, and histogram, right) and RORγ t+(black gate, left, and histogram, right) Treg cells isolated from the large intestine laminia propria. 6h, Percentage RFP+ cells among eGFP+CD4+ Thy1.1+(open circles) and Thy1.1−(black circles) cells in Tbx21RFP-creERT2RorcGFP/WT mice. LN, lymph node; SI, small intestine; LI, large intestine. 6i, Flow cytometry of CD4 T cells in Tbx21RFP-creERT2RorcGFP/WT mice as quantified in 6h. 6j, Top, RFP+(left axis, squares) and YFP+(right axis, circles) effector CD4 T cells. Bottom, percentage of RFP+ among YFP+ effector CD4 T cells 3 weeks (white symbols), 3 months (grey symbols), and 7 months (black symbols) after tamoxifen gavage, as outlined in FIG. 1b. Data are mean±s.e.m. All data are representative of ≥2 experiments, n≥4 mice per group each.

FIGS. 7a-7f demonstrate that T-bet^{lo} cells probably represent transient unstable intermediates in the differentiation of stable T-bethi Treg cells. 7a, Flow cytometry of the indicated cell subsets. 7b, CD44 and CD62L expression on RFP−CXCR3− (grey shaded histograms, squares), RFPloCXCR3− (black histograms, squares), and RFPhiCXCR3+(red histograms, squares) splenic CD4+ Thy1.1+ cells. 7c, Differential gene expression between CD44hiRFP− and CD44hiRFPhiCXCR3+ Treg cells sorted from pooled spleens and lymph nodes of Tbx21RFP-creERT2 mice. All genes significantly up—(red) or downregulated (blue) are indicated. 7d, Expression of the 288 genes up—(≥1.5-fold; left) or 184 genes downregulated (≤1.5-fold; right) in CD44hiRFPhiCXCR3+ versus CD44hiRFP−cells. Genes with a mean expression value of <15 were excluded from the analysis. P, paired t-test; adjustments were made for multiple comparisons. 7e, CD44loCD62LhiRFP−, CD44hiRFP−, CD44hiRFPloCXCR3−, and CD44hiRFPhiCXCR3hi CD4+ Thy1.1+ cells were FACS-sorted and transferred into lymphoreplete hosts and analysed in pooled spleens and lymph nodes 14 days after transfer. 7f, Quantification of data in e using a two-tailed t-test (***P<0.001). All data are representative of ≥2 experiments, n≥2 mice per group each.

FIGS. 8a-8j demonstrate fate mapping of T-bet-expressing T_{reg} cells during infectious challenge. 8a, Preferential expansion of CD44ₕᵢRFP-versus CD44ₕᵢRFP₊ CD4 effector T cells during N. brasiliensis infection. Flow cytometry analysis of splenic (top) and lung (bottom) CD4₊Thy1.1₋ cells from mice challenged with PBS (left) and N. brasiliensis (Nb, right). 8b, Flow cytometry of splenic CD4+ Thy1.1+(left) and Thy1.1₋ (right) cells of mice challenged with PBS (top) and L. monocytogenes (Lm, bottom), as indicated in FIG. 2a. Numbers indicate percentage of RFP+(left) and YFP+(right) cells. 8c, Top, schematic of experiment. CD44ₗₒCD62LₕᵢRFP−, CD44ₕᵢRFP−, and CD44ₕᵢRFPₕᵢCXCR3ₕᵢ CD4+ Thy1.1+ cells were FACS-sorted from pooled spleens and lymph nodes of Tbx21_{RFP-creERT2} mice and transferred into lymphoreplete hosts one day before PBS or L. monocytogenes challenge. Bottom, flow cytometry of transferred populations (indicated on left) on day 9 in spleens of mice challenged with PBS (left) or L. monocytogenes (right). 8d, Representative histograms of RFP and CXCR3 expression on total CD4₊Thy1.1₊ (shaded histograms) or Th1.1₊YFP₊(open histograms) cells from spleens of mice challenged with PBS (black) or L. monocytogenes (red), as indicted in FIG. 2a. 8e-8g, eGFP expression in PBS or L. monocytogenes challenged Tbx21_{RFP-creERT2}Il10_{eGFP} mice. 8e, Schematic of tamoxifen (Tx) administration to Tbx21_{RFP-creERT2}Il10_{eGFP/WT} mice for data shown in 8f, 8g. 8f, Flow cytometry of T_{reg} (top) and YFP₊ T_{reg} (bottom) cells in spleens of PBS (left) and L. monocytogenes (right) treated mice. 8g, Left, percentage of RFP-eGFP₊ and RFP₊eGFP₊ among T_{reg} cells, as gated in f (top). Right, percentage of eGFP₊ cells among YFP₊ T_{reg} cells, as gated in 8f (bottom). 8h, Schematic of L. monocytogenes reinfection in Tbx21_{RFP-creERT2}Il10_{eGFP/WT} mice for data shown in i, j; 1° and 2° indicate primary and secondary challenge, respectively. 8i, Flow cytometry of cells in Tbx21_{RFP-creERT2}Il10_{eGFP} mice on day 65, treated as indicated above. 8j, Percentage of RFP-eGFP₊ and RFP₊eGFP₊ cells among Thy1.1₊ cells, as gated in 8i. All data are representative of ≥2 experiments, n≥2 mice per group each. Data are mean±s.e.m. Two-tailed t-test (NS, not significant).

FIGS. 9a-9e demonstrates features of T-bet+ Treg cells. 9a, T cell activation, CXCR3 expression, and cytokine production in 12-week-old control Foxp3YFP-creTbx21WT/WT and Foxp3YFP-creTbx21fl/WT (white circles) and experimental Foxp3YFP-creTbx21fl/fl (black circles) mice. Data are mean±s.e.m. Two-tailed t-test (*P<0.05; NS, not significant). Data are representative of three experiments, n≥7 mice per group. 9b, Cumulative distribution function plot of the 561 genes up in Thy1.1+CD44hiRFPhiCXCR3+ versus CD44hiRFP− cells in Tbx21RFP-creERT2 mice compared to all genes differentially expressed in CD4+CD25+ Treg cells from Tbx21RFP-creFoxp3WT mice versus CD4+CD2510 ex-Treg cells from Tbx21RFP-creFoxp3fl mice. P=0.2×10-15, two-sample Kolmogorov-Smirnov test. 9c, Expression of CCR5 (top) and CD29 (bottom) in CD44loCD62Lhi naive (blue histogram), CD44hiCXCR3− (black histogram) and CD44hiCXCR3+(red histogram) Treg (left) and CD4+Foxp3− (right) T cells from spleens of Foxp3YFP-creTbx21WT/WT mice. 9d, Expression of CXCR3 (left), CCR5 (middle), and CD29 (right), gated on CD4 T cells in spleens of Foxp3YFP-creTbx21WT/WT and Foxp3YFP-creTbx21fl/fl mice. 9e, Dendrogram represents cluster analysis of TCR sequences in CD44hiCXCR3+(red symbols) and CD44hiCXCR3− (black symbols) Treg (right) and effector CD4 T (left) cells in spleens (white symbols) and lymph nodes (grey symbols) of DO11.10 TCRβ+Tcra+/−Foxp3 reporter mice. Sample preparation and statistical analyses are described in the Methods. Pearson's correlation of clonotype frequencies for the shared TCR clones was used for the generation of the dendrogram.

FIGS. 10a-10h shows characterization of Tbx21RFP-creFoxp3fl mice. 10a, Targeting strategy for the Tbx21 locus (top) and RFP expression in the indicated cell populations in spleens of homozygous Tbx21RFP-cre mice (bottom). 10b, Progressive loss of hair pigmentation in Tbx21RFP-creFoxp3fl mice. 10c, RFP and YFP expression (upper) and CD44 and CD62L expression (lower) in the indicated splenic cell populations in Tbx21RFP-creR26Y mice. 10d, Activation and expansion of RFP+ T cells in lymph nodes (top) and lungs (bottom) of the indicated mice. 10e, Cytokine production by CD4+Foxp3− and CD8+ T cells in lungs of the indicated mice. 10f, Characterization of lymph node Treg cells. 10g, Percentages of ex-Treg cells in spleens, lymph nodes, and lungs. 10h, Top, flow cytometry of lymph node CD4 T cells, as quantified in g; numbers indicate the percentage of Foxp3−CD25+. Bottom, histogram showing expression of Treg cell signature molecules in CD4+Foxp3−CD25+ cells in lymph nodes of Tbx21RFP-creFoxp3WT (open grey histogram), Tbx21RFP-creFoxp3fl (open red histogram), Tbx21RFP-creFoxp3WT/WT (open blue histogram), and Tbx21RFP-cre Foxp3fl/WT (open black histogram) mice. CD4+Foxp3+CD25+ cells from a Tbx21RFP-creFoxp3WT (shaded grey histogram) mouse are shown as a point of reference. Data are mean±s.e.m. Two-tailed t-test (***P<0.001, **P<0.01 and *P<0.05, respectively; NS, not significant). Data represent the combined results from several experiments.

FIGS. 11a-11d demonstrate that a TH2 response to N. brasiliensis is not exacerbated in Tbx21RFP-creFoxp3fl mice. Tbx21RFP-creFoxp3fl and Tbx21RFP-creFoxp3WT mice were infected with N. brasiliensis and analysed on day 9 after challenge. 11a, Flow cytometry of GATA3 expression in CD4+Foxp3−CD25− T cells in spleens (top) and lungs (bottom) of Tbx21RFP-creFoxp3WT (left) and Tbx21RFP-creFoxp3fl (right) mice. 11b, Quantification of data in a. Tbx21RFP-creFoxp3WT and Tbx21RFP-creFoxp3fl mice are indicated by grey and red circles, respectively. 11c, Numbers of eosinophils in lungs of the indicated mice. 11d, Cytokine production by CD4+Foxp3− and CD8 T cells in spleens and lungs of the indicated mice. Data are mean±s.e.m. Two-tailed t-test (*P<0.05; NS, not significant). Data represents 1 experiment, n≥5 mice per group.

FIGS. 12a-12e Distinguishes the drivers of autoimmunity in the absence of T-bet+ Treg cells. 12a-12c, Ex-Treg cells are no more pathogenic than effector CD4 T cells. a, CD4+CD25+(Treg) cells were sorted from lymph nodes of Tbx21RFP-creFoxp3WT mice, and CD4+CD25− (effector) and CD4+CD2510 (ex-Treg) cells were sorted from lymph nodes of Tbx21RFP-cre Foxp3fl mice for transfer into Tcrb−/−Tcrd−/−mice. Intracellular staining for Foxp3 demonstrates purity of cell populations. 12b, Weights of Tcrb−/−Tcrd−/− mice receiving CD4+CD25+(white squares), CD4+CD25− (black squares), and CD4+CD2510 (grey squares) cells. 12c, Percentages and numbers of the indicated T cell populations in spleens of mice analysed on day 62 after transfer. 12d, 12e, T-bet+ effector αβT cells drive disease upon ablation of T-bet+ Treg cells. Lethally irradiated Tcrb−/−Tcrd−/− mice were reconstituted with a 1:1 mix of CD45.2+Tbx21RFP-cre/WTR26iDTR T-cell depleted bone marrow cells with either CD45.1+Foxp3KO, CD45.1+Foxp3WT, or CD45.2+TcrbKO T-cell depleted bone marrow cells. Mice were injected with 0.5 μg diphtheria toxin (DT) on day 0, then treated daily with 0.1 μg diphtheria toxin for 22 days before analysis. 12d, Weight loss in Tbx21RFP-cre/WTR26iDTR:Foxp3KO (red line) versus Tbx21RFP-cre/WT R26iDTR:Foxp3WT (black line) versus Tbx21RFP-cre/WTR26iDTR:TcrbKO (blue line) reconstituted mice. 12e, Representative flow cytometry of splenic cell populations (indicated on right) in chimaeric mice (as indicated above). All data represent 1 experiment, n≥3 mice per group.

FIGS. 13a-13f show co-localization of T-bet+ Treg and T-bet+ effector T cells in vivo. 13a, 13b, Representative images (left) and insets (right) of lymph node sections from Tbx21RFP-cre mice with CD4 (a) or CD8 (b) in green, RFP in red, Foxp3 in blue, and CD44 in grey. In inset, arrowheads indicate CD4+CD44hiRFP+Foxp3− (13a) or CD8+CD44hiRFP+(13b) cells and arrows indicate CD4+CD44hiRFP+Foxp3+ cells. 13c, 13d, Quantification of nearest distances between Treg cells and CD4 (13c) and CD8 (13d) T cells, as shown in 13a, 13b. Foxp3+ denotes CD4+CD44hiFoxp3+; Foxp3− (13c) denotes CD4+CD44hiFoxp3− and CD8+(13d) denotes CD8+CD44hiRFP+. 13e, 13f, Quantification of nearest distances between Treg and non-Treg CD4 (13e) and CD8 (130 T cells in spleens of Tbx21RFP-creFoxp3WT and Tbx21RFP-cre Foxp3fl mice. Genotypes of mice are indicated above plots; cell types being analysed are shown below plots, as in c, d. Bars indicate mean. P values were calculated using a two-tailed t-test (13c, 13d) or one-way ANOVA (13e, 13f) (***P<0.001, **P<0.01, *P<0.05; NS, not significant). Data are representative of multiple imaged sections from ≥2 mice.

FIGS. 14a-14i|T-bet+ Treg cells suppress pre-established TH1 but not TH2 or TH17 activation induced by depletion of Treg cells. 14a, Targeting strategy for the Foxp3 locus. 14b, Schematic for experiment shown in 14c-14g depleting all Treg cells and subsequently depleting all or only non-T-bet-expressing Treg cells in Foxp3fl-DTRTbx21RFP-creERT2 mice. 14c, Flow cytometry of splenic CD4 T cells in the indicated mice treated with tamoxifen or oil, as indicated. 14d-14g, Percentages of Treg cells (14d) and activation status of (14e) and cytokine production by (14f, 14g) splenic CD4+Foxp3− and CD8 T cells in tamoxifen-treated Foxp3Thy1.1 Tbx21RFP-creERT2 (open circles), mock oil-treated Foxp3fl-DTRTbx21RFP-creERT2 (black circles), and tamoxifen-treated Foxp3fl-DTRTbx21RFP-creERT2 (grey circles) mice. 14h-14l, Treg cells rebounding post depletion in DT-treated Foxp3DTRTbx21RFP-creERT2 mice efficiently suppress TH2 responses. 14h, Left, schematic for control experiment shown in 14i-14l. Right, flow cytometry of splenic CD4 T cells in mice treated with high dose diphtheria toxin (DThi, 1μ, g per mouse), low dose diphtheria toxin (DTlo, 0.0625 μg per mouse), and PBS. Group 1 (control); group 2 (depletion without Treg cell recovery); group 3 (depletion with partial recovery); group 4 (depletion with full recovery). 14i-14l, Percentages of Treg cells (14i) and activation status of (14j) and cytokine production by (14k, 14l) splenic CD4+Foxp3− and CD8 T cells in the indicated groups of mice. Data are mean±s.e.m. Two-tailed t-test (***P<0.001, **P<0.01, *P<0.05; NS, not significant). Data are representative of ≥1 experiment, n≥4 mice per group.

FIGS. 15a-15e demonstrate Treg cells rebounding post transient depletion efficiently suppress TH2 and TH17 responses. 15a, Experimental schematic. Mice were treated with tamoxifen (tx) or oil (to additionally control for potential effects of tamoxifen) on days −5 and −3 and received PBS on days 0, 1, 3, 5, 7 (control); 1 μg diphtheria toxin (DThi) on days 0, 1, 3, 5, and 7 (no Treg cell recovery); 0.062 μg diphtheria toxin (DTlo) on days 0, 1, 3, 5, and 7 (partial Treg cell recovery); or 0.062 μg diphtheria toxin (DTlo) on day 0 and PBS on days 1, 3, 5, and 7 (full Treg cell recovery). Mice were analysed on day 9. 15b, Flow cytometry analysis of CD4 T cells in spleens of the indicated groups of mice. 15c-15e, Percentages of Treg cells (15c) and CD4+Foxp3− and CD8 T cell activation (15d) and cytokine production (15e) in spleens of the indicate mice (group 1, open circles; group 2, black circles; group 3, dark grey circles; group 4, light grey circles). Data are mean±s.e.m. Two-tailed t-test (***P<0.001, **P<0.01; NS, not significant). Data represent the combined results from two experiments, n≥3 mice per group.

### DEFINITIONS

Administration: As used herein, the term “administration” refers to the administration of a composition to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (including by intratracheal instillation), transdermal, vaginal and vitreal. In some embodiments, administration may be intratumoral or peritumoral. In some embodiments, administration may involve intermittent dosing. In some embodiments, administration may involve continuous dosing (e.g., perfusion) for at least a selected period of time.

Adoptive cell therapy: As used herein, “adoptive cell therapy” or “ACT” involves the transfer of immune cells, e.g Tregs, into subjects. In some embodiments, ACT is a treatment approach that involves the use of lymphocytes with regulatory T-cell activity, the in vitro expansion of these cells to large numbers and their infusion into a subject.

Agent: The term “agent” as used herein may refer to a compound or entity of any chemical class including, for example, polypeptides, nucleic acids, saccharides, lipids, small molecules, metals, or combinations thereof. As will be clear from context, in some embodiments, an agent can be or comprise a cell or organism, or a fraction, extract, or component thereof. In some embodiments, an agent is or comprises a natural product in that it is found in and/or is obtained from nature. In some embodiments, an agent is or comprises one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents are provided as collections or libraries, for example that may be screened to identify or characterize active agents within them. Some particular embodiments of agents that may be utilized in accordance with the present invention include small molecules, antibodies, antibody fragments, aptamers, nucleic acids (e.g., siRNAs, shRNAs, DNA/RNA hybrids, antisense oligonucleotides, ribozymes), peptides, peptide mimetics, etc. In some embodiments, an agent is or comprises a polymer. In some embodiments, an agent is not a polymer and/or is substantially free of any polymer. In some embodiments, an agent contains at least one polymeric moiety. In some embodiments, an agent lacks or is substantially free of any polymeric moiety.

Allergen: The term “allergen”, as used herein, refers to those antigens that induce an allergic reaction. In some embodiments, an allergen is or comprises a polypeptide. In some embodiments, an allergen is or comprises a small molecule. In some embodiments, an allergen is selected from the group consisting of food allergens, drug allergens, environmental allergens, insect venoms, animal allergens, and latex.

Allergic reaction: The phrase “allergic reaction,” as used herein, has its art-understood meaning and refers to an IgE-mediated immune response to an antigen. When an antigen induces IgE antibodies, they will bind to IgE receptors on the surface of basophils and mast cells. Subsequent exposures to the antigen trigger cross-linking of such surface-bound anti-allergen IgEs, which trigger release of histamine from stores within the cells. This histamine release triggers the allergic reaction. Typically, an allergic reaction involves one or more of the cutaneous (e.g., uticana, angiodema, pruritus), respiratory (e.g., wheezing, coughing, laryngeal edema, rhinorrhea, watery/itching eyes), gastrointestinal (e.g., vomiting, abdominal pain, diarrhea), and/or cardiovascular (e.g., if a systemic reaction occurs) systems. For the purposes of the present invention, an asthmatic reaction is considered to be a form of allergic reaction. In some embodiments, allergic reactions are mild; typical symptoms of a mild reaction include, for example, hives (especially over the neck and face) itching, nasal congestion, rashes, watery eyes, red eyes, and combinations thereof. In some embodiments, allergic reactions are severe and/or life threatening; in some embodiments, symptoms of severe allergic reactions (e.g., anaphylactic reactions) are selected from the group consisting of abdominal pain, abdominal breathing sounds (typically high-pitched), anxiety. chest discomfort or tightness, cough, diarrhea, difficulty breathing, difficulty swallowing, dizziness or light-headedness, flushing or redness of the face, nausea or vomiting, palpitations, swelling of the face, eyes or tongue, unconsciousness, wheezing, and combinations thereof. In some embodiments, allergic reactions are anaphylactic reactions.

Allergy: The term “allergy”, as used herein, refers to a condition characterized by an IgE-mediated immune response to particular antigens. In some embodiments, the antigens are ones that do not elicit an IgE-mediated immune response in many or most individuals. In some embodiments, the term “allergy” is used to refer to those situations where an individual has a more dramatic IgE-mediated immune response when exposed to a particular antigen than is typically observed by members of the individual's species when comparably exposed to the same antigen. Thus, an individual who is suffering from or susceptible to “allergy” is one who experiences or is at risk of experiencing an allergic reaction when exposed to one or more allergens. In some embodiments, symptoms of allergy include, for example, presence of IgE antibodies, reactive with the allergen(s) to which the individual is allergic, optionally above a particular threshold, in blood or serum of the individual. In some embodiments, symptoms of allergy include development of a wheel/flare larger than a control wheel/flare when a preparation of the antigen is injected subcutaneously under the individual's skin. In some embodiments, an individual can be considered susceptible to allergy without having suffered an allergic reaction to the particular allergen in question. For example, if the individual has suffered an allergic reaction, and particularly if the individual has suffered an anaphylactic reaction, to a related allergen (e.g., one from the same source or one for which shared allergies are common), that individual may be considered susceptible to allergy to (and/or to an allergic or anaphylactic reaction to) the relevant allergen. Similarly, if members of an individual's family react to a particular allergen, the individual may be considered to be susceptible to allergy to (and/or to an allergic and/or anaphylactic reaction to) that allergen.

Amelioration: As used herein, “amelioration” refers to prevention, reduction and/or palliation of a state, or improvement of the state of a subject. Amelioration includes, but does not require, complete recovery or complete prevention of a disease, disorder or condition.

Amino acid: As used herein, term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally occurring amino acid. In some embodiments, an amino acid is a synthetic amino acid; in some embodiments, an amino acid is a d-amino acid; in some embodiments, an amino acid is an 1-amino acid. “Standard amino acid” refers to any of the twenty standard 1-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or posttranslational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are composed of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present disclosure include glycosylated Fc domains, including Fc domains with modified or engineered such glycosylation. For purposes of the present disclosure, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are fully human, or are humanized, primatized, chimeric, etc, as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present disclosure is in a format selected from, but not limited to, intact IgG, IgE and IgM, bi- or multi-specific antibodies (e.g., Zybodies®, etc), single chain Fvs, polypeptide-Fc fusions, Fabs, cameloid antibodies, masked antibodies (e.g., Probodies®), Small Modular ImmunoPharmaceuticals (“SMIPsTM”), single chain or Tandem diabodies (TandAb®), VHHs, Anticalins®, Nanobodies®, minibodies, BiTE®s, ankyrin repeat proteins or DARPINs®, Avimers®, a DART, a TCR-like antibody, Adnectins®, Affilins®, Trans-Bodies®, Affibodies®, a TrimerX®, MicroProteins, Fynomers®, Centyrins®, and a KALBITOR®. In some embodiments, an antibody may lack a covalent modification (e.g., attachment of a glycan) that it would have if produced naturally. In some embodiments, an antibody may contain a covalent modification (e.g., attachment of a glycan, a payload (e.g., a detectable moiety, a therapeutic moiety, a catalytic moiety, etc.), or other pendant group (e.g., poly-ethylene glycol, etc.)).

Antigen: The term “antigen”, as used herein, refers to an agent that elicits an immune response; and/or an agent that binds to a T cell receptor (e.g., when presented by an MHC molecule) or to an antibody or antibody fragment. In some embodiments, an antigen elicits a humoral response (e.g., including production of antigen-specific antibodies); in some embodiments, an antigen elicits a cellular response (e.g., involving T-cells whose receptors specifically interact with the antigen). In some embodiments, an antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (in some embodiments other than a biologic polymer (e.g., other than a nucleic acid or amino acid polymer)) etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source), or alternatively may exist on or in a cell. In some embodiments, an antigen is a recombinant antigen.

Antigen presenting cell: The phrase “antigen presenting cell” or “APC,” as used herein, has its art understood meaning referring to cells that process and present antigens to T-cells. Exemplary APC include dendritic cells, macrophages, B cells, certain activated epithelial cells, and other cell types capable of TCR stimulation and appropriate T cell costimulation.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated: Two events or entities are “associated” with one another, as that term is used herein, if the presence, level, degree, type and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Binding: It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell).

Carrier: as used herein, refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step

Determine: Many methodologies described herein include a step of “determining”. Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Dosage form: As used herein, the terms “dosage form” and “unit dosage form” refer to a physically discrete unit of a therapeutic agent for the patient to be treated. Each unit contains a predetermined quantity of active material calculated to produce the desired therapeutic effect. It will be understood, however, that the total dosage of the composition will be decided by the attending physician within the scope of sound medical judgment.

Dosing regimen: As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Engineered: Those of ordinary skill in the art, reading the present disclosure, will appreciate that the term “engineered”, as used herein, refers to an aspect of having been manipulated and altered by the hand of man. In particular, the term “engineered cell” refers to a cell that has been subjected to a manipulation, so that its genetic, epigenetic, and/or phenotypic identity is altered relative to an appropriate reference cell such as otherwise identical cell that has not been so manipulated. In some embodiments, the manipulation is or comprises a genetic manipulation. In some embodiments, a genetic manipulation is or comprises one or more of (i) introduction of a nucleic acid not present in the cell prior to the manipulation (i.e., of a heterologous nucleic acid); (ii) removal of a nucleic acid, or portion thereof, present in the cell prior to the manipulation; and/or (iii) alteration (e.g., by sequence substitution) of a nucleic acid, or portion thereof, present in the cell prior to the manipulation. In some embodiments, an engineered cell is one that has been manipulated so that it contains and/or expresses a particular agent of interest (e.g., a protein, a nucleic acid, and/or a particular form thereof) in an altered amount and/or according to altered timing relative to such an appropriate reference cell. In some embodiments, an “engineered cell” refers to a cell that has been isolated and/or cultured under controlled conditions, for example so that a population of cells (i.e., an “engineered cell population” or a “population of engineered cells”) defined by characteristics that result from such isolation and/or culturing is obtained or provided. Those of ordinary skill in the art will appreciate that reference to an “engineered cell” herein may, in some embodiments, encompass both the particular cell to which the manipulation was applied and also any progeny of such cell.

Excipient: as used herein, refers to a non-therapeutic agent that may be included in a pharmaceutical composition, for example to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Gene: As used herein, the term “gene” has its meaning as understood in the art. It will be appreciated by those of ordinary skill in the art that the term “gene” may include gene regulatory sequences (e.g., promoters, enhancers, etc.) and/or intron sequences. It will further be appreciated that definitions of gene include references to nucleic acids that do not encode proteins but rather encode functional RNA molecules such as tRNAs, RNAi-inducing agents, etc. For the purpose of clarity we note that, as used in the present application, the term “gene” generally refers to a portion of a nucleic acid that encodes a protein; the term may optionally encompass regulatory sequences, as will be clear from context to those of ordinary skill in the art. This definition is not intended to exclude application of the term “gene” to non-protein—coding expression units but rather to clarify that, in most cases, the term as used in this document refers to a protein-coding nucleic acid.

Gene product or expression product: As used herein, the term “gene product” or “expression product” generally refers to an RNA transcribed from the gene (pre- and/or post-processing) or a polypeptide (pre- and/or post-modification) encoded by an RNA transcribed from the gene.

Heterologous: As used herein, the term “heterologous” refers to an agent (e.g. a nucleic acid, protein, cell, tissue, etc) that is present in a particular context as a result of engineering as described herein (i.e., by application of a manipulation to the context). To give but a few examples, a nucleic acid or protein that is ordinarily or naturally found in a first cell type and not in a second cell type (e.g., in a bacterial cell and not in a mammalian cell, in a cell from a first tissue and not in a cell from a second tissue, in a cell of a first microbial species but not in a cell of a second microbial species, etc) may be “heterologous” to the second cell type. Analogously, a cell or tissue that is ordinarily or naturally found in a first organism and not in a second organism (e.g., in a rodent and not in a mammal, etc) may be “heterologous” to the second organism. Those of ordinary skill in the art will understand the scope and content of the term “heterologous” as used herein.

Immune response: As used herein, the term “immune response” refers to a response elicited in an animal. In some embodiments, an immune response may refer to cellular immunity, humoral immunity or may involve both. In some embodiments, an immune response may be limited to a part of the immune system. For example, in certain embodiments, an immune response may be or comprise an increased IFNγ response. In certain embodiments, immune response may be or comprise mucosal IgA response (e.g., as measured in nasal and/or rectal washes). In certain embodiments, an immune response may be or comprise a systemic IgG response (e.g., as measured in serum). In certain embodiments, an immune response may be or comprise a neutralizing antibody response. In certain embodiments, an immune response may be or comprise a cytolytic (CTL) response by T cells. In certain embodiments, an immune response may be or comprise reduction in immune cell activity.

Improve, increase, or reduce: As used herein, the terms “improve,” “increase” or “reduce,” or grammatical equivalents, indicate values that are relative to an appropriate reference measurement, as will be understood by those of ordinary skill in the art. To give but a few examples, in some embodiments, application of such a term in reference to an individual who has received a particular treatment may indicate a change relative to a comparable individual who has not received the treatment, and/or to the relevant individual him/herself prior to administration of the treatment, etc.

Individual, subject: As used herein, the terms “subject” or “individual” refer to a particular human or non-human mammalian organism; in many embodiments, the terms refer to a human. In some embodiments, an “individual” or “subject” may be a member of a particular age group (e.g., may be a fetus, infant, child, adolescent, adult, or senior). In some embodiments, an “individual” or “subject” may besuffering from or susceptible to a particular disease, disorder or condition (i.e., may be a “patient”).

In vitro: The term “in vitro” as used herein refers to events that occur in an artificial environment, e.g., in a test tube or reaction vessel, in cell culture, etc., rather than within a multi-cellular organism.

In vivo: as used herein refers to events that occur within a multi-cellular organism, such as a human and a non-human animal. In the context of cell-based systems, the term may be used to refer to events that occur within a living cell (as opposed to, for example, in vitro systems).

Isolated: as used herein, refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced.

Nucleic acid: As used herein, “nucleic acid”, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to individual nucleic acid residues (e.g., nucleotides and/or nucleosides); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is single stranded; in some embodiments, a nucleic acid is double stranded. In some embodiments a nucleic acid has a nucleotide sequence comprising at least one element that encodes, or is the complement of a sequence that encodes, a polypeptide. In some embodiments, a nucleic acid has enzymatic activity.

Patient: As used herein, the term “patient” refers to a organism who is suffering from or susceptible to a disease, disorder or condition and/or who will receive administration of a diagnostic, prophylactic, and/or therapeutic regimen. In many embodiments, a patient displays one or more symptoms of a disease, disorder or condition. In some embodiments, a patient has been diagnosed with one or more diseases, disorders or conditions. In some embodiments, the disorder or condition is or includes cancer, or presence of one or more tumors. In some embodiments, a patient is receiving or has received certain therapy to diagnose, prevent (i.e., delay onset and/or frequency of one or more symptoms of) and/or to treat a disease, disorder, or condition.

Peptide: The term “peptide” as used herein refers to a polypeptide that is typically relatively short, for example having a length of less than about 100 amino acids, less than about 50 amino acids, less than 20 amino acids, or less than 10 amino acids.

Pharmaceutically acceptable: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Protein: As used herein, the term “protein”, refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof.

Reference: As used herein, “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Response: As used herein, a response to treatment may refer to any beneficial alteration in a subject's condition that occurs as a result of or correlates with treatment. Such alteration may include stabilization of the condition (e.g., prevention of deterioration that would have taken place in the absence of the treatment), amelioration of symptoms of the condition, and/or improvement in the prospects for cure of the condition, etc. It may refer to a subject's response or to a tumor's response. Tumor or subject response may be measured according to a wide variety of criteria, including clinical criteria and objective criteria. Techniques for assessing response include, but are not limited to, clinical examination, positron emission tomatography, chest X-ray CT scan, MRI, ultrasound, endoscopy, laparoscopy, presence or level of tumor markers in a sample obtained from a subject, cytology, and/or histology. Many of these techniques attempt to determine the size of a tumor or otherwise determine the total tumor burden. Methods and guidelines for assessing response to treatment are discussed in Therasse et. al., “New guidelines to evaluate the response to treatment in solid tumors”, European Organization for Research and Treatment of Cancer, National Cancer Institute of the United States, National Cancer Institute of Canada, J. Natl. Cancer Inst., 2000, 92(3):205-216. The exact response criteria can be selected in any appropriate manner, provided that when comparing groups of tumors and/or patients, the groups to be compared are assessed based on the same or comparable criteria for determining response rate. One of ordinary skill in the art will be able to select appropriate criteria.

Specialized: The term “specialized” as used herein, refers to a composition, agent, entity or population that has acquired certain functional and/or phenotypic characteristics, e.g., as a result of conditions of its preparation and/or development.

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, or condition (e.g., cancer) has been diagnosed with and/or exhibits one or more symptoms of the disease, disorder, or condition.

Susceptible to: An individual who is “susceptible to” a disease, disorder, and/or condition is one who has a higher risk of developing the disease, disorder, and/or condition than does a member of the general public. In some embodiments, an individual who is susceptible to a disease, disorder and/or condition may not have been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Symptoms are reduced: According to the present invention, “symptoms are reduced” when one or more symptoms of a particular disease, disorder or condition is reduced in magnitude (e.g., intensity, severity, etc.) or frequency. For purposes of clarity, a delay in the onset of a particular symptom is considered one form of reducing the frequency of that symptom. It is not intended that the present invention be limited only to cases where the symptoms are eliminated. The present invention specifically contemplates treatment such that one or more symptoms is/are reduced (and the condition of the subject is thereby “improved”), albeit not completely eliminated.

T cell receptor: The terms “T cell receptor” or “TCR” are used herein in accordance with the typical understanding in the field, in reference to antigen-recognition molecules present on the surface of T-cells. During normal T-cell development, each of the four TCR genes, α, β, γ, and δ, can rearrange, so that T cells of a particular individual typically express a highly diverse population of TCR proteins.

Therapeutic agent: As used herein, the phrase “therapeutic agent” in general refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

Therapeutically effective amount: As used herein, the term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, stabilizes one or more characteristics of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. For example, in some embodiments, “therapeutically effective amount” refers to an amount which, when administered to an individual in need thereof in the context of inventive therapy, will block, stabilize, attenuate, or reverse a cancer-supportive process occurring in said individual, or will enhance or increase a cancer-suppressive process in said individual. In the context of cancer treatment, a “therapeutically effective amount” is an amount which, when administered to an individual diagnosed with a cancer, will prevent, stabilize, inhibit, or reduce the further development of cancer in the individual. A particularly preferred “therapeutically effective amount” of a composition described herein reverses (in a therapeutic treatment) the development of a malignancy such as a pancreatic carcinoma or helps achieve or prolong remission of a malignancy. A therapeutically effective amount administered to an individual to treat a cancer in that individual may be the same or different from a therapeutically effective amount administered to promote remission or inhibit metastasis. As with most cancer therapies, the therapeutic methods described herein are not to be interpreted as, restricted to, or otherwise limited to a “cure” for cancer; rather the methods of treatment are directed to the use of the described compositions to “treat” a cancer, i.e., to effect a desirable or beneficial change in the health of an individual who has cancer. Such benefits are recognized by skilled healthcare providers in the field of oncology and include, but are not limited to, a stabilization of patient condition, a decrease in tumor size (tumor regression), an improvement in vital functions (e.g., improved function of cancerous tissues or organs), a decrease or inhibition of further metastasis, a decrease in opportunistic infections, an increased survivability, a decrease in pain, improved motor function, improved cognitive function, improved feeling of energy (vitality, decreased malaise), improved feeling of well-being, restoration of normal appetite, restoration of healthy weight gain, and combinations thereof. In addition, regression of a particular tumor in an individual (e.g., as the result of treatments described herein) may also be assessed by taking samples of cancer cells from the site of a tumor such as a pancreatic adenocarcinoma (e.g., over the course of treatment) and testing the cancer cells for the level of metabolic and signaling markers to monitor the status of the cancer cells to verify at the molecular level the regression of the cancer cells to a less malignant phenotype. For example, tumor regression induced by employing the methods of this invention would be indicated by finding a decrease in one or more pro-angiogenic markers, an increase in anti-angiogenic markers, the normalization (i.e., alteration toward a state found in normal individuals not suffering from cancer) of metabolic pathways, intercellular signaling pathways, or intracellular signaling pathways that exhibit abnormal activity in individuals diagnosed with cancer. Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

Transformation: As used herein, “transformation” refers to any process by which exogenous DNA is introduced into a host cell. Transformation may occur under natural or artificial conditions using various methods well known in the art. Transformation may rely on any known method for the insertion of foreign nucleic acid sequences into a prokaryotic or eukaryotic host cell. In some embodiments, a particular transformation methodology is selected based on the host cell being transformed and may include, but is not limited to, viral infection, electroporation, mating, lipofection. In some embodiments, a “transformed” cell is stably transformed in that the inserted DNA is capable of replication either as an autonomously replicating plasmid or as part of the host chromosome. In some embodiments, a transformed cell transiently expresses introduced nucleic acid for limited periods of time.

Treatment: As used herein, the term “treatment” (also “treat” or “treating”) refers to any administration of a substance that partially or completely alleviates, ameliorates, relives, inhibits, delays onset of, reduces severity of, and/or reduces incidence of one or more symptoms, features, and/or causes of a particular disease, disorder, and/or condition (e.g., cancer). Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Vector: as used herein, refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.”

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose.

### DETAILED DESCRIPTION OF CERTAIN EMBODIMENTS

The present invention provides, among other things, compositions and methods relating to modified and/or specialized regulatory T-cells (Treg) or cell populations, and their use in the treatment of various diseases, disorders, and conditions. Specifically, the present invention contemplates the use of engineered Tregs for the treatment of autoimmune and/or inflammatory diseases.

The present disclosure demonstrates, among other things, that in Treg cells expression of the T_{H}1-associated transcription factor T-bet, induced at steady state and following infection, gradually becomes highly stable even under non-permissive conditions. In some embodiments, loss-of-function or elimination of T-bet-expressing Treg cells, but not of T-bet itself, resulted in a severe T_{H}1-type autoimmunity. In some embodiments, after selective depletion of T-bet-negative Treg cells, the remaining T-bet-expressing cells inhibited specifically T_{H}1 and CD8⁺ T cell activation in agreement with pronounced co-localization with T-bet⁺ effector CD4⁺ and CD8⁺ T cells.

### Adaptive Immune Responses

The adaptive immune system comprises several different cell types which can mount or cause different responses when activated by respective antigens. The cell types include antigen presenting cells (APCs), effector or helper cells, and regulator cells. Effector cells differentiate from Naive CD4+ T cells into functionally distinct subsets such as T helper 1 (T_{H}1) and T helper 2 (T_{H}2) defined by expression of key transcription factors T-bet and GATA3, respectively.

The Th1 response is characterized by the production of IFN-γ which activates the activities of macrophages, and induces B cells to make opsonizing (coating) and complement-fixing antibodies, and leads to cell-mediated immunity. In general, Th1 responses are more effective against intracellular pathogens.

T box expressed in T cells (Tbet), a T box transcription factor, is generally recognized as regulating expression of Th1 hallmark cytokines, e.g. IFN-γ, and driving differentiation of naïve CD4+ T-cells towards a Th1 lineage.

The Th2 response is characterized by the release of Interleukin 5, which induces eosinophils in the clearance of parasites. Th2 also produce Interleukin 4, which facilitates B cell isotype switching. In general, Th2 responses are more effective against extracellular bacteria, parasites including helminths and toxins.

GATA binding protein 3 (GATA-3) is generally recognized as regulating expression of Th2 hallmark cytokines, e.g. IFN-γ, and driving differentiation of naïve CD4+ T-cells towards a Th2 lineage.

### Regulatory T Cells

Regulatory T cells (Tregs) are important in maintaining homeostasis, controlling the magnitude and duration of the inflammatory response, and in preventing autoimmune and allergic responses.

The Forkhead box P3 transcription factor (Foxp3) has been shown to be a key regulator in the differentiation and activity of Treg. In fact, loss-of-function mutations in the Foxp3 gene have been shown to lead to the lethal IPEX syndrome (immune dysregulation, polyendocrinopathy, enteropathy, X-linked). Patients with IPEX suffer from severe autoimmune responses, persistent eczema, and colitis. Regulatory T (Treg) cells expressing transcription factor Foxp3 play a key role in limiting inflammatory responses in the intestine (Josefowicz, S. Z. et al. Nature, 2012, 482, 395-U1510).

In general Tregs are thought to be mainly involved in suppressing immune responses, functioning in part as a “self-check” for the immune system to prevent excessive reactions. In particular, Tregs are involved in maintaining tolerance to self-antigens, harmless agents such as pollen or food, and abrogating autoimmune disease.

Tregs are found throughout the body including, without limitation, the gut, skin, lung, and liver. Additionally, Treg cells may also be found in certain compartments of the body that are not directly exposed to the external environment such as the spleen, lymph nodes, and even adipose tissue. Each of these Treg cell populations is known or suspected to have one or more unique features and additional information may be found in Lehtimaki and Lahesmaa, Regulatory T cells control immune responses through their non-redundant tissue specific features, 2013, FRONTIERS IN IMMUNOL., 4(294): 1-10, the disclosure of which is hereby incorporated in its entirety.

### Cell Engineering

Those skilled in the art are aware of a wide variety of technologies available for engineering of cells (e.g., mammalian cells, and particularly mammalian Treg cells). For example, various systems for introducing nucleic acids for expression in and/or integration into such cells are well known in the art, as are various strategies for achieving epigenetic modification of cells.

In some embodiments, cell engineering technologies appropriate for use in accordance with the present disclosure may be or comprise introduction of one or more heterologous nucleic acids into a cell. In some embodiments, technologies for introduction of a heterologous nucleic acid into a cell include, among other things, transfection, electroporation including nucleofection, and transduction. Various vector systems for introduction of heterologous nucleic acids are known in the art, including but not limited to, plasmids, bacterial artificial chromosomes, yeast artificial chromosomes, and viral systems (e.g, adenoviruses and lentiviruses).

In some embodiments, cell engineering technologies appropriate for use in accordance with the present disclosure may be or comprise introduction of one or more heterologous proteins into a cell. In some embodiments, technologies for introduction of a heterologous protein into a cell include, among other things, transfection, transduction with cell permeable peptides (e.g. TAT), and nanoparticle delivery.

In general, cells may be engineered as described herein so that they express a transcription factor of interest (i.e., so that level and/or activity of an active form of a transcription factor of interest is increased in the cell). In some embodiments a transcription factor of interest is one that regulates differentiation of T-cells. In some embodiments a transcription factor of interest is, for example, Tbet or GATA3.

Those of ordinary skill in the art will appreciate that a variety of engineering strategies could achieve such increased expression. For example, to name but a few, in some embodiments, a transcription factor of interest may be introduced; a protein inducing the expression of a transcription factor of interest may be introduced, a protein increasing the stability of a transcription factor of interest may be introduced, or a protein reducing the degradation of a transcription factor of interest may be introduced.

In some embodiments, a introduced nucleic acid may be or comprise a sequence that encodes, or is complimentary to a nucleic acid that encodes, part or all of a transcription factor of interest. In some embodiments, an introduced nucleic acid may be or comprise a regulatory sequence functional in the cell to regulate expression of a nucleic acid that encodes, or is complimentary to a nucleic acid that encodes, part or all of a transcription factor of interest.

In some embodiments, the methods and compositions of the present disclosure relate to the use of a subjects own, or autologous, cells. In some embodiments, the methods and compositions of the present disclosure relate to the use of heterologous cells. The methods and compositions of the present disclosure are relevant to the engineering Treg cells for the treatment of various diseases, disorders and conditions.

In some embodiments, engineering strategies as provided herein achieve a regulatory T-cell or population of cells that has increased expression of a transcription factor of interest relative to a reference. In some embodiments, a regulatory T-cell is characterized by 2, 5, 10, 20, 30, 40, 50, 60, 70, 80, 90 fold greater expression of an mRNA for a transcription factor of interest relative to a reference. In some embodiments, a regulatory T-cell is characterized by 2, 5, 10, 20, 30, 40, 50, 60, 70, 80, 90 fold greater expression of a transcription factor of interest relative to a reference. In some embodiments, a regulatory T-cell is characterized by about 10-20, 20-30, 30-40, 40-50, 50 to 60, 60 to 70, 70 to 80, 80 to 90 fold greater expression of an mRNA for a transcription factor of interest relative to a reference. In some embodiments, a regulatory T-cell is characterized by about 10-20, 20-30, 30-40, 40-50, 50 to 60, 60 to 70, 70 to 80, 80 to 90 fold greater expression of a transcription factor of interest relative to a reference.

In some embodiments, a reference is the expression level of a transcription factor of interest in a regulatory T-cell from the source (e.g., an individual donor) of the engineered regulatory T-cell. In some embodiments, a reference is the expression level of a transcription factor of interest in a population of individuals. In some embodiments, a reference is the expression level of a transcription factor of interest in a is a historical standard.

In some embodiments, engineering strategies as provided herein achieve a regulatory T-cell or population of cells characterized by a particular level (e.g., elevated level) of expression of one or more markers of interest. In some such embodiments, such a marker may be a cell surface marker, an intracellular marker, and/or a secreted marker. In some embodiments, a regulatory T-cell or population of cells is characterized by 2, 5, 10, 20, 30, 40, 50, 60 fold greater expression of such a marker. For example, in some embodiments, the present disclosure provides a cultured population of Treg cells characterized by 10, 20, 30, 40, 50, 60, 70, 80, 90, 100% of the population expressing Tbet.

### Diseases, Disorders, and Conditions

In some embodiments, methods and compositions of the present disclosure are relevant to the treatment of, among other things, diseases, disorders or conditions characterized by inflammation. In some embodiments, methods and compositions of the present disclosure are relevant to the treatment of, among other things, diseases, disorders or conditions characterized by autoimmunity. In some embodiments, methods and compositions of the present disclosure are relevant to the treatment of, among other things, diseases, disorders or conditions characterized by a Th1 response. In some embodiments, methods and compositions of the present disclosure are relevant to the treatment of, among other things, diseases, disorders or conditions characterized by a Th2 response.

Inflammation

Inflammation, as used herein, refers to the localized protective response of vascular tissues to injury, irritation or infection. Inflammatory conditions are characterized by one or more of the following symptoms: redness, swelling, pain and loss of function. Inflammation is a protective attempt by the organism to remove the harmful stimuli and begin the healing process. Although infection is caused by a microorganism, inflammation is one of the responses of the organism to the pathogen.

Inflammation can be classified as either acute or chronic. Acute inflammation is the initial response of the body to harmful stimuli and is achieved by the increased movement of plasma and leukocytes (especially granulocytes) from the blood into the injured tissues. A cascade of biochemical events propagates and matures the inflammatory response, involving the local vascular system, the immune system, and various cells within the injured tissue. Prolonged inflammation, known as chronic inflammation, leads to a progressive shift in the type of cells present at the site of inflammation and is characterized by simultaneous destruction and healing of the tissue from the inflammatory process.

Inflammation may be caused by a number of agents, including infectious pathogens, toxins, chemical irritants, physical injury, hypersensitive immune reactions, radiation, foreign irritants (dirt, debris, etc.), frostbite, and burns. Transplanted or transfused tissues, organs or blood products, among other things, can also be included in the broad category of foreign irritants. Graft versus host disease is one example of a disease, disorder, or condition arising from inflammation from transplanted or transfused tissues, organs or blood products. Types of inflammation include colitis (e.g., ulcerative colitis), inflammatory bowel disease (e.g. Crohn's disease), bursitis, appendicitis, dermatitis, cystitis, rhinitis, tendonitis, tonsillitis, vasculitis, and phlebitis.

Autoimmunity

Autoimmunity refers to the presence of a self-reactive immune response (e.g., auto-antibodies, self-reactive T-cells). Autoimmune diseases, disorders, or conditions arise from autoimmunity through damage or a pathologic state arising from an abnormal immune response of the body against substances and tissues normally present in the body. Damage or pathology as a result of autoimmunity can manifest as, among other things, damage to or destruction of tissues, altered organ growth, and/or altered organ function.

Types of autoimmune diseases, disorders or conditions include type I diabetes, alopecia areata, vasculitis, temporal arteritis, rheumatoid arthritis, lupus, celiac disease, Sjogrens syndrome, polymyalgia rheumatica, and multiple sclerosis.

In some embodiments, the present disclosure contemplates a population of engineered regulatory T cells characterized by an ability to suppress an immune response. In some embodiments a population of engineered regulatory T cells is characterized by an ability to alleviate a disease, a disorder or condition. In some embodiments a population of engineered regulatory T cells is characterized by an ability to alleviate a disease, a disorder or condition by suppression of an immune response. In some embodiments a population of engineered regulatory T cells is characterized by an ability to reduce inflammation. In some embodiments a population of engineered regulatory T cells is characterized by an ability to suppress an autoimmune response.

### Administration

Certain embodiments of the disclosure include administration of an engineered regulatory T-cell to a subject; or a composition comprising of an engineered regulatory T-cell.

In some embodiments, a regulatory T-cell is obtained from a subject and modified and/or cultured as described herein to obtain an engineered regulatory T-cell. Thus, in some embodiments, an engineered regulatory T-cell comprises an autologous cell that is administered into the same subject from which an immune cell was obtained. Alternatively, an immune cell is obtained from a subject and is transformed, e.g., transduced, as described herein, to obtain an engineered regulatory T-cell that is allogenically transferred into another subject.

In some embodiments, a regulatory T-cell for use in accordance with the present disclosure is obtained by collecting a sample from a subject containing immune cells and isolating regulatory T-cells from the sample. In some embodiments, a regulatory T-cell for use in accordance with the present disclosure is obtained by collecting a sample from a subject containing immune cells and isolating an immune cell sub-population (e.g. CD4+ cells, CD8+ cells, etc.) for use in in vitro generation of regulatory T-cells. In some embodiments, a regulatory T-cell for use in accordance with the present disclosure is obtained by collecting a sample from a subject containing immune cells and isolating naïve CD4+ T-cells for use in for in vitro generation of regulatory T-cells. In some embodiments, a regulatory T-cell for use in accordance with the present disclosure is obtained by collecting a sample from a subject containing immune cells and isolating naïve CD8+ T-cells for use in for in vitro generation of regulatory T-cells.

Those skilled in the art are aware of a wide variety of techniques available for in vitro generation of regulatory T-cell. For example, activation of isolated immune cells with plate-bound anti-CD3 and soluble anti-CD28 in the presence of TGF-β, IFN-γ, and/or IL-27.

In some embodiments, an engineered regulatory T-cell is autologous to a subject, and the subject can be immunologically naive, immunized, diseased, or in another condition prior to isolation of an immune cell from the subject.

In some embodiments, additional steps can be performed prior to administration of an engineered regulatory T-cell to a subject. For instance, an engineered regulatory T-cell can be expanded in vitro after modification. In vitro expansion can proceed for 1 day or more, e.g., 2 days or more, 3 days or more, 4 days or more, 6 days or more, or 8 days or more, prior to the administration to a subject. Alternatively, or in addition, in vitro expansion can proceed for 21 days or less, e.g., 18 days or less, 16 days or less, 14 days or less, 10 days or less, 7 days or less, or 5 days or less, prior to administration to a subject. For example, in vitro expansion can proceed for 1-7 days, 2-10 days, 3-5 days, or 8-14 days prior to the administration to a subject.

In some embodiments, during in vitro expansion, an engineered regulatory T-cell can be stimulated with an antigen (e.g., a TCR antigen). Antigen specific expansion optionally can be supplemented with expansion under conditions that non-specifically stimulate lymphocyte proliferation such as, for example, anti-CD3 antibody, anti-Tac antibody, anti-CD28 antibody, ionomycin and/or phytohemagglutinin (PHA). The expanded engineered regulatory T-cell can be directly administered into a subject or can be frozen for future use, i.e., for subsequent administrations to a subject.

In some embodiments, within a population of engineered regulatory T-cells 100%, 90%, 80%, 70%, 60%, 50%, 40%, 30%, or 20% of the Tregs express the transcription factor of interest (e.g. Tbet).

In certain embodiments, an engineered regulatory T-cell is administered prior to, substantially simultaneously with, or after administration of another therapeutic agent. An engineered regulatory T-cell described herein can be formed as a composition, e.g., an engineered regulatory T-cell and a pharmaceutically acceptable carrier. In certain embodiments, a composition is a pharmaceutical composition comprising at least one engineered regulatory T-cell described herein and a pharmaceutically acceptable carrier, diluent, and/or excipient. Pharmaceutically acceptable carriers described herein, for example, vehicles, adjuvants, excipients, and diluents, are well-known and readily available to those skilled in the art. Preferably, the pharmaceutically acceptable carrier is chemically inert to the active agent(s), e.g., an engineered regulatory T-cell, and does not elicit any detrimental side effects or toxicity under the conditions of use.

A composition can be formulated for administration by any suitable route, such as, for example, intravenous, intratumoral, intraarterial, intramuscular, intraperitoneal, intrathecal, epidural, and/or subcutaneous administration routes. Preferably, the composition is formulated for a parenteral route of administration.

A composition suitable for parenteral administration can be an aqueous or nonaqueous, isotonic sterile injection solution, which can contain anti-oxidants, buffers, bacteriostats, and solutes, for example, that render the composition isotonic with the blood of the intended recipient. An aqueous or nonaqueous sterile suspension can contain one or more suspending agents, solubilizers, thickening agents, stabilizers, and preservatives.

Dosage administered to a subject, particularly a human, will vary with the particular embodiment, the composition employed, the method of administration, and the particular site and subject being treated. However, a dose should be sufficient to provide a therapeutic response. A clinician skilled in the art can determine the therapeutically effective amount of a composition to be administered to a human or other subject in order to treat or prevent a particular medical condition. The precise amount of the composition required to be therapeutically effective will depend upon numerous factors, e.g., such as the specific activity of the engineered regulatory T-cell, and the route of administration, in addition to many subject-specific considerations, which are within those of skill in the art.

Any suitable number of engineered regulatory T-cells can be administered to a subject. While a single engineered regulatory T-cell described herein is capable of expanding and providing a therapeutic benefit, in some embodiments, 10² or more, e.g., 10³ or more, 10⁴ or more, 10⁵ or more, or 10⁸ or more, engineered regulatory T-cells are administered. Alternatively, or additionally 10¹² or less, e.g., 10¹¹ or less, 10⁹ or less, 10⁷ or less, or 10⁵ or less, engineered regulatory T-cells described herein are administered to a subject. In some embodiments, 10²-10⁵, 10⁴-10⁷, 10³-10⁹, or 10⁵-10¹⁹ engineered regulatory T-cells described herein are administered.

A dose of an engineered regulatory T-cell described herein can be administered to a mammal at one time or in a series of subdoses administered over a suitable period of time, e.g., on a daily, semi-weekly, weekly, bi-weekly, semi-monthly, bi-monthly, semi-annual, or annual basis, as needed. A dosage unit comprising an effective amount of an engineered regulatory T-cell may be administered in a single daily dose, or the total daily dosage may be administered in two, three, four, or more divided doses administered daily, as needed.

Route of administration can be parenteral, for example, administration by injection, transnasal administration, transpulmonary administration, or transcutaneous administration. Administration can be systemic or local by intravenous injection, intramuscular injection, intraperitoneal injection, subcutaneous injection.

### EXEMPLIFICATION

### Example 1: Methods

Animals.

Tbx2ltdTomato-T2A-creERT2 mice were generated by insertion of a targeting construct into the Tbx21 locus by homologous recombination in embryonic stem cells on the C57BL/6 background; the targeting construct was generated by inserting sequence containing exons 2-5 of the Tbx21 gene from BAC RP23-237M14 (BACPAC Resources Center) into a plasmid backbone containing a PGK promoter driving expression of diphtheria toxin A subunit (DTA) followed by BGHpA sequence (modified PL452 plasmid). A SalI restriction enzyme site was simultaneously engineered into the Tbx21 3′ UTR between the stop codon and the polyadenylation site. The Clontech Infusion HD Cloning system was used to generate in the pUC19 plasmid backbone sequence containing (in order from 5′ to 3′) encephalomyocarditis virus IRES; tandem dimer (td) Tomato; T2A self-cleaving peptide from Thosea asigna virus; Cre recombinase fused to the oestrogen receptor ligand binding domain (ER); followed by a frt site-flanked PGK-Neomycin resistance gene (NEO)-BGHpA cassette. The IRES-tdTomato-T2A-CreERT2-frt-NEOBGHpA-frt sequence was PCR-amplified and inserted into the SalI site in the Tbx21 3′ UTR in the modified PL452 backbone. The resulting plasmid was linearized with the restriction enzyme NotI before electroporation into embryonic stem cells. Tbx2ltdTomato-T2A-cre mice were generated similarly, with Cre recombinase containing a nuclear localization sequence replacing the CreERT2 sequence. Tbx2ltdTomato-T2A-creERT2 and Tbx2ltdTomato-T2A-cre mice were bred to FLPeR mice to excise the NEO cassette and backcrossed to C57BL/6 mice to remove the FLPeR allele.

Foxp3fl-DTR mice were similarly generated by insertion of a targeting construct into the Foxp3 locus by homologous recombination in embryonic stem cells on the C57BL/6 background; the targeting construct was generated by inserting sequence containing exons 8-13 of the Foxp3 gene from a 30.8-kb cosmid containing the complete Foxp3 gene into the plasmid backbone containing a PGK promoter driving expression of diphtheria toxin A subunit (DTA) followed by BGHpA sequence (modified PL452 plasmid). The Clontech Infusion HD Cloning system was used to generate in the pUC19 plasmid backbone sequence containing (in order from 5′ to 3′) a loxP site; encephalomyocarditis virus IRES; diptheria toxin receptor (DTR) enhanced green fluorescent protein (eGFP) fusion protein; a triple SV40 polyA (STOP); a second loxP site; encephalomyocarditis virus IRES; Thy1.1; followed by a frt site-flanked PGK-Neomycin resistance gene (NEO)-BGHpA cassette. The loxP-IRES-DTReGFP-STOP-loxP-IRES-Thy1.1-frt-NEO-BGHpA-frt sequence was PCR-amplified and inserted into the BaeI site in the Foxp3 3′ UTR in the modified PL452 backbone. The resulting plasmid was linearized with the restriction enzyme NotI before electroporation into embryonic stem cells. Foxp3fl-DTR mice were bred to FLPeR mice to excise the NEO cassette and backcrossed to C57BL/6 mice to remove the FLPeR allele. Foxp3Thy1.1, R26Y, Foxp3fl, Foxp3KO, RorcGFP, Foxp3YFP-cre, IL-10eGFP, and Tbx21fl mice have been previously described^{2,20-25}. CD45.1, R26iDTR, and TcrbKO mice were purchased from Jackson Laboratories²⁶. Foxp3Thy1.1Tbx21CreERT2R26Y (called Tbx21CreERT2 mice in the text) mice are homozygous at each locus. Tbx21RFP-cre Foxp3WT, Tbx21RFP-creFoxp3fl, Tbx21RFP-creFoxp3WT/WT, and Tbx21RFP-creFoxp3WT/fl mice described in the text are homozygous for the Tbx21 knock-in allele. Foxp3fl-DTRTbx21RFP-creERT2 mice described in the text are homozygous at each locus. Generation and treatments of mice were performed under protocol 08-10-023 approved by the Sloan Kettering Institute (SKI) Institutional Animal Care and Use Committee. All mouse strains were maintained in the SKI animal facility in specific pathogen free (SPF) conditions in accordance with institutional guidelines and ethical regulations. For tamoxifen administration, 40 mg tamoxifen dissolved in 100 μl ethanol and subsequently in 900 μl olive oil (Sigma-Aldrich) were sonicated 4×30 s in a Bioruptor Twin (Diagenode). Mice were orally gavaged with 200 μl tamoxifen emulsion per treatment. For diphtheria toxin (DT) injections, DT (Sigma-Aldrich) was dissolved in PBS and 200 μl of indicated doses (1 μg per mouse unless otherwise indicated) were injected i.p. per mouse. For antibiotic treatment, mice were weaned onto filtered antibiotic-treated water containing ampicillin, kanamycin, vancomycin and metronidazole (0.1% w/v each). All mice analysed were sex and aged matched (8-12 weeks old) with the exception of some Tbx21RFP-creFoxp3WT and Tbx21RFP-creFoxp3fl mice used for immunofluorescence imaging that were up to 10 months of age (results were similar to in 8-12-week-old mice).

Isolation of Cells.

For analysis of YFP-labelled CD4 T cells in Tbx21RFP-creERT2 mice, CD4 T cells in spleens and lymph nodes were enriched using the Dynabeads CD4 Positive Isolation Kit (Invitrogen). To isolate lymphocytes from tissues, mice were euthanized and immediately perfused with 20 ml PBS. Small and large intestines were removed, flushed with PBS and Peyer's patches were removed. Subsequently, 0.5-cm-long fragments of intestines were washed in PBS and incubated in PBS supplemented with 5% fetal calf serum, 1% 1-glutamine, 1% penicillin—streptomycin, 10 mM HEPES, 1 mM dithiothreitol, and 1 mM EDTA for 15 min. Samples were washed and incubated in digest solution (RPMI supplemented with 5% fetal calf serum, 1% 1-glutamine, 1% penicillin—streptomycin, 10 mM HEPES, 1 mg ml-1 collagenase, and 1 U ml-1 DNase I) for 10 min twice. After filtering through a 100-μm strainer, cells were resuspended in 35% Percoll to eliminate debris. Lymphocytes from livers and lungs were isolated by 50-60 min incubation in digest solution, filtered through 100-μm strainers, and after debris removal in 35% Percoll, purified by centrifugation (1,000 g, 7.5 min) over a step-wise 44%/67% Percoll gradient at room temperature.

Nippostrongylus brasiliensis and Listeria monocytogenes Infections.

N. brasiliensis was maintained by passage in 9-10-week-old male Wistar rats as previously described²⁷. In brief, rats were injected subcutaneously (s.c.) with 7000 L3 N. brasiliensis and stool was collected on days 6-9 after infection. Fecal pellets were mixed with 5×8 bone charcoal and incubated on moist filter paper in Petri dishes at 26° C. for 7 days. L3 larvae were recovered from the edge of the filter paper and the perimeter of the plates and extensively washed with PBS to eliminate contaminants before infection. Mice infections were carried out using a 23 G needle at a concentration of 500 L3 N. brasiliensis in 200 μl. For L. monocytogenes infections, frozen stocks were thawed, resuspended in Brain-Heart Infusion media, and grown at 37° C. to an OD600 of 0.1. For primary infections, mice were injected via lateral tail vein with 5-10×103 colony-forming units (cfu) of L. monocytogenes diluted in 200 μl PBS. For secondary infection, mice were injected via lateral tail vein with 105 cfu of L. monocytogenes in 200 μl PBS. Treatments of rats were performed under protocol 08-10-023 approved by the Sloan Kettering Institute (SKI) Institutional Animal Care and Use Committee. Rats were maintained in the SKI animal facility in Biosafety Level 2 conditions in accordance with institutional guidelines and ethical regulations.

Cell Transfer Experiments.

For cell transfer experiments, pooled spleens and lymph nodes were enriched for CD4 T cells using the Dynabeads CD4 Positive Isolation Kit. Cells were FACS-sorted on an Aria II cell sorter (BD Bioscience), washed 3 times in PBS, resuspended in 200 μl PBS, and transferred into recipients via retro-orbital injection.

Generation of Bone Marrow Chimaeric Mice.

Tcrb−/−Tcrd−/−recipient mice were lethally irradiated with 650 Gy. The following day, bone marrow was isolated from femurs of donor mice and depleted of T cells and RBCs via staining with biotinylated anti-Thy1.2 and anti-Ter119 antibodies followed by magnetic bead negative selection. 5×106 total T-cell-depleted bone marrow cells were transferred into recipient mice via retro-orbital injection.

Flow cytometric analysis. Cells were stained with LIVE/DEAD Fixable Yellow Dead Cell Stain (Molecular Probes) and the following antibodies purchased from eBioscience, BioLegend, BD Biosciences, Tonbo, or obtained from the NIH tetramer core facility: anti-CD4 (RM4-5, Biolegend 100548), anti-CD8a (5H10, BD Biosciences 564297), anti-TCRβ (H57-597, eBioscience 47-5961-82), PBS-57-loaded mCD1d tetramer (NIH 26181), anti-Thy1.1 (HIS51, eBioscience 17-0900-82), anti-CD44 (IM7, BioLegend 103026), anti-CD62L (MEL-14, eBioscience 25-0621-82), anti-CXCR3 (CXCR3-173, eBioscience 17-1831-173), anti-CD25 (PC61.5, eBioscience 17-0251), anti-CTLA-4 (UC10-4B9, eBioscience 17-1522-82), anti-GITR (DTA-1, eBioscience 48-5874-82), anti-CD39 (24-DMS1, eBioscience 25-0391-82), anti-CD11b (M1/70, Tonbo Bioscience 25-01120U100), anti-SiglecF (E50-2440, BD Pharmingen 562681), anti-CCR5 (HM-CCR5(7A4) (eBioscience 12-1951-82) and C34-3448 (BD Biosciences 559921), anti-CD29 (eBioHMb1-1, eBioscience 48-0291-80), anti-Foxp3 (FJK-16 s, Tonbo Bioscience 35-5773-U100), anti-T-bet (4B10, BioLegend 644816), anti-RORγ t (B2D, eBioscience 12-6981-82), anti-Gata-3 (TWAJ, eBioscience 46-9966-41), anti-DsRed (Living Colours DsRed Polyclonal Antibody, Clontech 632496), anti-IFNγ (XMG1.2, eBioscience 48-7311-80), anti-IL-4 (11B11, eBioscience 51-7041-82), anti-IL-17A (17B7, eBioscience 61-7177-82), anti-IL-13 (eBiol3A, eBioscience 12-7133-82), anti-IL-5 (BD Pharmingen, 554396), and anti-IL-2 (JES6-5H4, eBioscience 25-7021-82). Flow cytometric analysis was performed using an LSRII flow cytometer (BDBioscience) and FlowJo software (Tree Star). Intracellular staining was performed using eBioscience Fixation Permeabilization buffers. For cytokine staining lymphocytes were stimulated with soluble anti-CD3 clone 2C11 (5 μg ml-1) and anti-CD28 clone 37.51 (5 μg ml-1) in the presence of 1 μg ml-1 brefeldin A for 5 h at 37° C., 5% CO2. Unless otherwise stated, CD4 T cells were pre-gated as TCRβ+PBS-57-CD1d tetramer-cells.

RNA-Seq Analysis.

Pooled spleens and lymph nodes were enriched for CD4 T cells using the Dynabeads CD4 Positive Isolation Kit. CD4+ Thy1.1+ cells were FACS-sorted on an Aria II cell sorter (BD Bioscience) into four populations (CD62LhiCD44loRFP−, CD44hiRFP−, CD44hiRFPloCXCR3-, and CD44hiRFPhiCXCR3+ cells) and resuspended in Trizol. Three replicates of each cell subset were generated. RNA-sequencing reads were aligned to the reference mouse genome GRCm38 using the Burrows-Wheeler Aligner (BWA)²⁸ and local realignment was performed using the Genome Analysis Toolkit (GATK)²⁹. For each sample, raw count of reads per gene was measured using R, and DESeq2 R package³⁰ was used to perform differential gene expression among different conditions. A cutoff of 0.05 was set on the obtained P values (that were adjusted using Benjamini-Hochberg multiple testing correction) to get the significant genes of each comparison.

TCR Sequencing and Data Analysis.

In brief, following isolation of CD4+ T cells from spleens and lymph nodes of DO11.10 TCRβ transgenic Tcra+/−Foxp3DTR mice using the Dynabeads CD4 Positive Isolation Kit (Invitrogen), CD44hiCXCR3− and CD44hiCXCR3+eGFP(Foxp3)+ Treg and eGFP−effector CD4 T cells were FACS sorted and stored in Trizol. TCR sequencing and data analysis were performed as previously described³¹. Pearson's correlation of clonotype frequencies for the shared TCR clones was used for the generation of the dendrogram.

Microscopy.

Confocal imaging was done using standard conditions. In brief, mice were perfused in PLP buffer. Lymph nodes and spleens were excised, fixed for 1 h at room temperature in 4% paraformaldehyde, and dehydrated at 4° C. in sucrose (30% in PBS). Tissues were snap-frozen in OCT compound (Sakura Tissue-Tek). 10 μm tissue sections were cut and fixed with Acetone for 20 min at −20° C., rehydrated in PBS and blocked with 10% normal donkey serum, in PBS with 0.3% Triton X-100, followed by overnight antibody staining at 4° C. in a humidified chamber. After antibody staining nuclei were stained with 5 μM Draq7 (Abcam) for 20 min at room temperature. Sections were imaged in Prolong Diamond mounting media (Life Technologies). All images were acquired using a confocal microscope (LSM880; Carl Zeiss) with a 40× oil immersion objective. Images were processed and analysed using ImageJ software (version 2.0.0-rc-54/1.51h; National Institutes of Health). Nearest neighbour analysis was performed using MATLAB (version R2016b, MathWorks).

Statistical Analysis.

All statistical analyses (excluding RNA-seq and TCR sequence analyses, described above) were performed using GraphPad Prism 6 software. Differences between individual groups were analysed for statistical significance using the unpaired or paired two-tailed t-test. *P≤0.05; **P≤0.01; ***P≤0.001; NS, not significant. The Kolmogorov-Smirnov test is used to determine the significance between the distributions of signature genes and the rest of expressed genes. One-way ANOVA is used to compare the means of three or more samples. No statistical method was used to predetermine sample size. The number of mice used in each experiment to reach statistical significance was determined on the basis of preliminary data. No animals were excluded from the analyses. No methods of randomization were used to allocate animals into experimental groups. No blinding was used. Data met assumptions of statistical methods used and variance was similar between groups that were statistically compared.

Code Availability.

The colocalization program (ImageJ software, 2.0.0-rc-54/1.51h, National Institutes of Health) was used to find cell positions and the MATLAB program (software R2016b, MathWorks) was used to calculate nearest cell distance.

Data Availability.

The RNA-seq data that support the findings of this study have been deposited in the NIH SRA database with the accession code SRP102941.

### Example 2: Stability and Function of Regulatory T Cells Expressing the Transcription Factor T-Bet

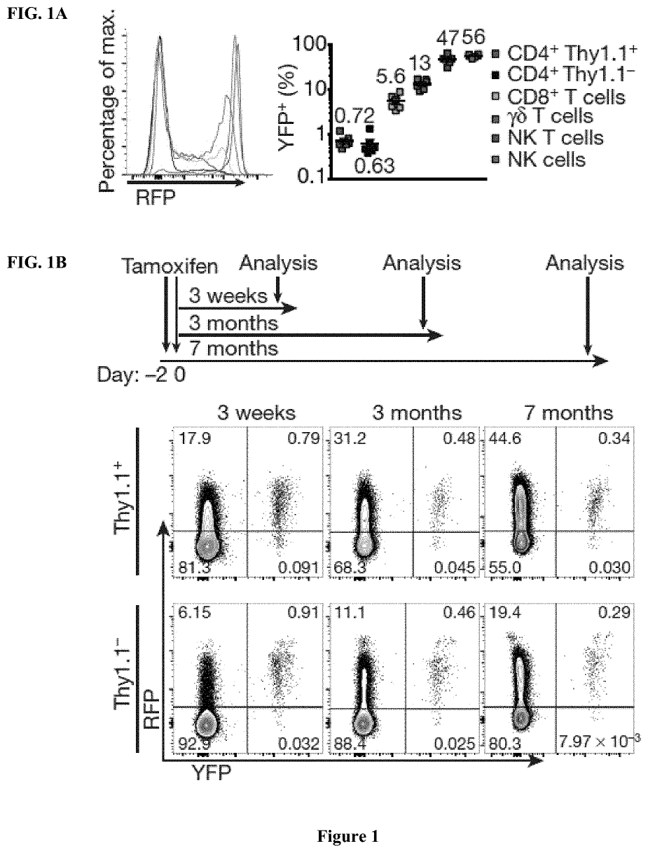

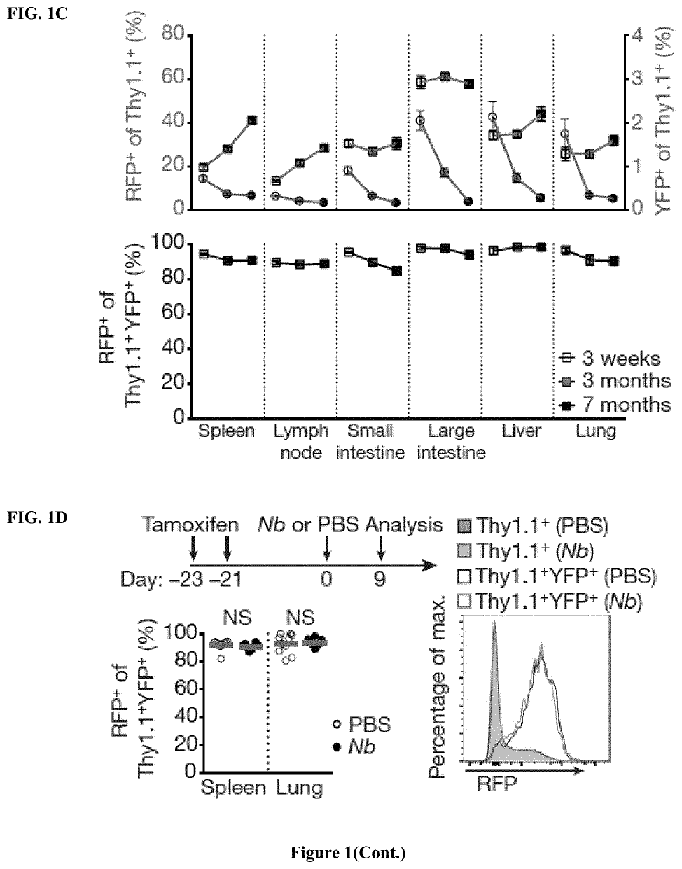

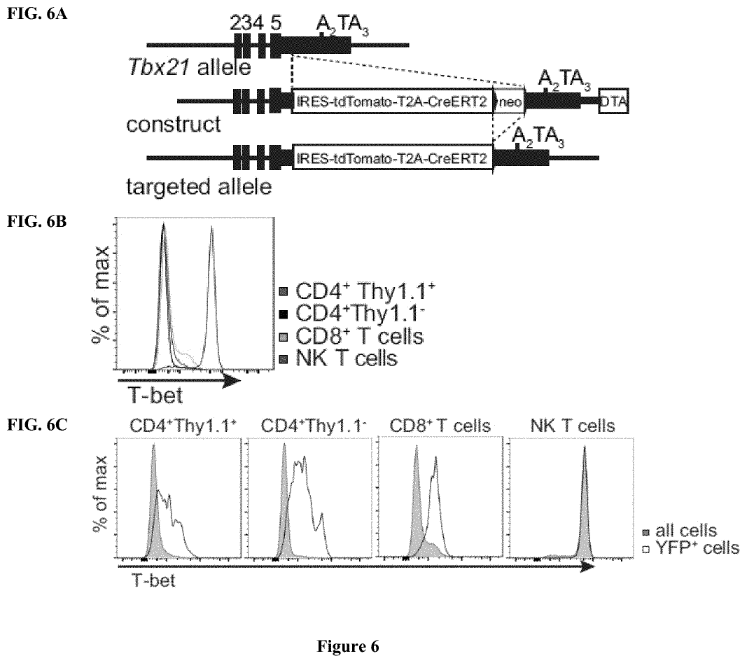

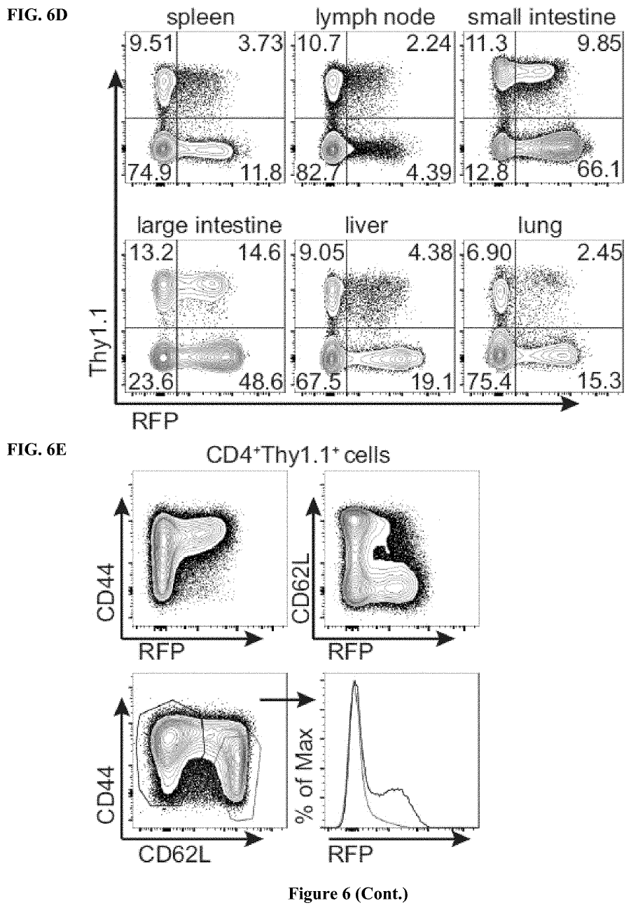

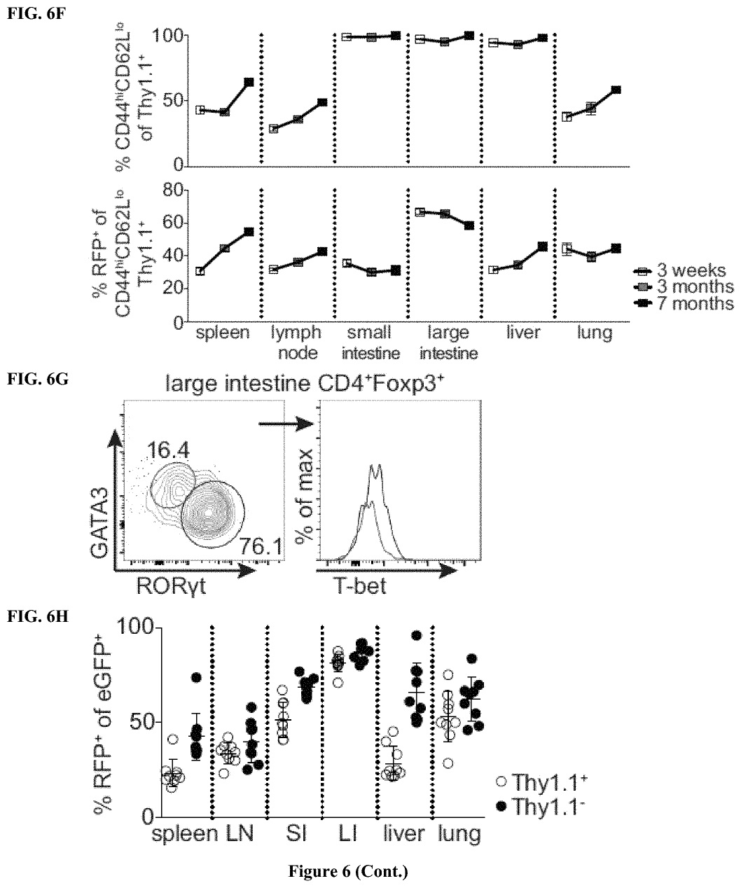

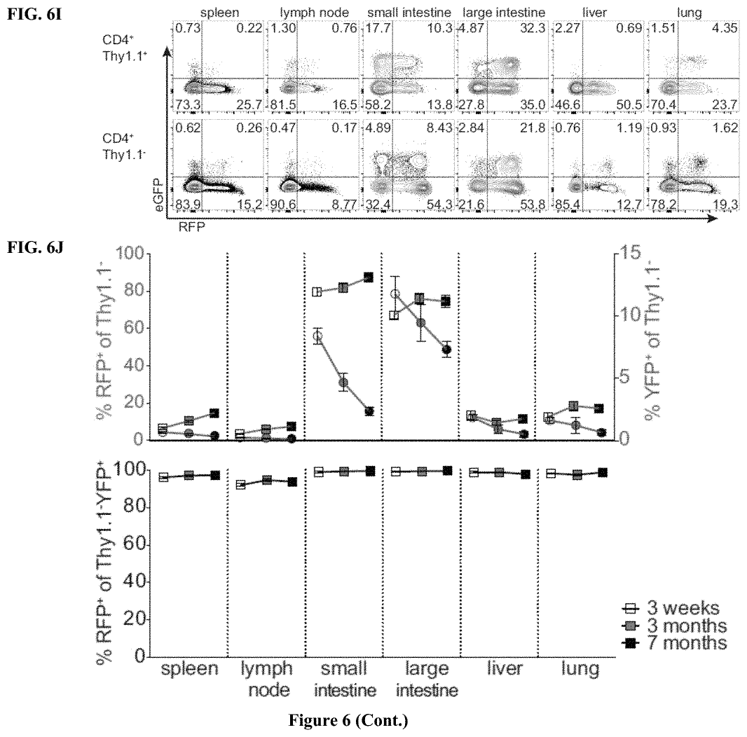

Whether Treg cells expressing the TH1-associated transcription factor T-bet represent a stable sub-lineage of cells with unique function or a transient activation state remains unknown. To address this question, we assessed the stability of T-bet expression in Treg cells using a novel Tbx21tdTomato-T2A-creERT2 knock-in allele combined with the R26Y recombination and Foxp3Thy1.1 reporters. The resulting Tbx21RFP-creERT2 mice showed a range of red fluorescent protein (RFP) expression and CreERT2 activity, which faithfully reflected endogenous T-bet protein levels in major lymphocyte subsets (FIG. 1a, FIG. 6a, 6b). RFP+ Treg cells comprised between 30-70% of CD44hiCD62Llo effector Treg cells in lymphoid organs and non-lymphoid tissues; interestingly, intestinal Treg cells exhibited prevalent co-expression of T-bet and ROR γ t, but not T-bet and GATA3 (FIG. 6d-6i).

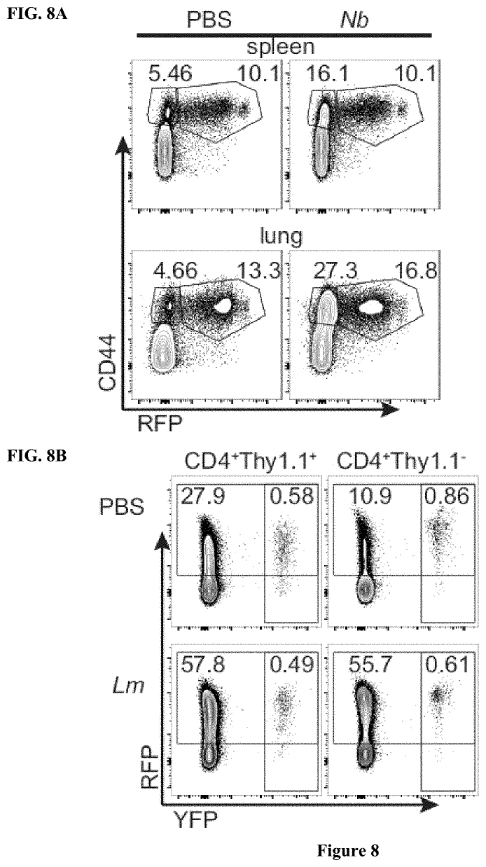

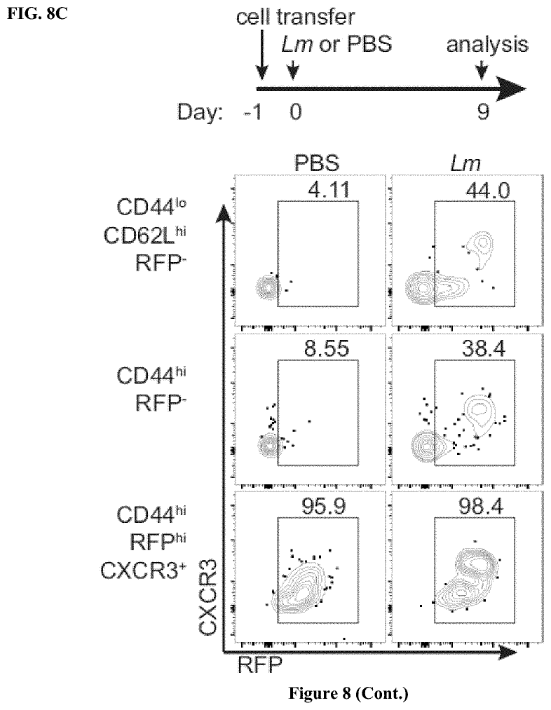

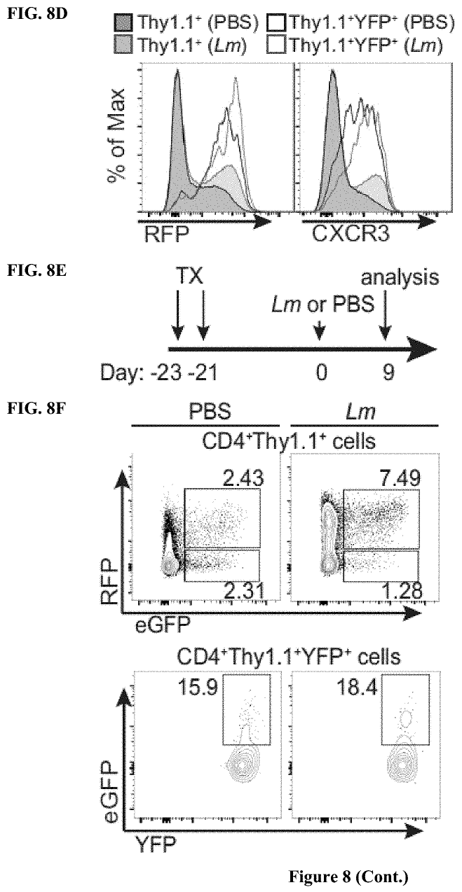

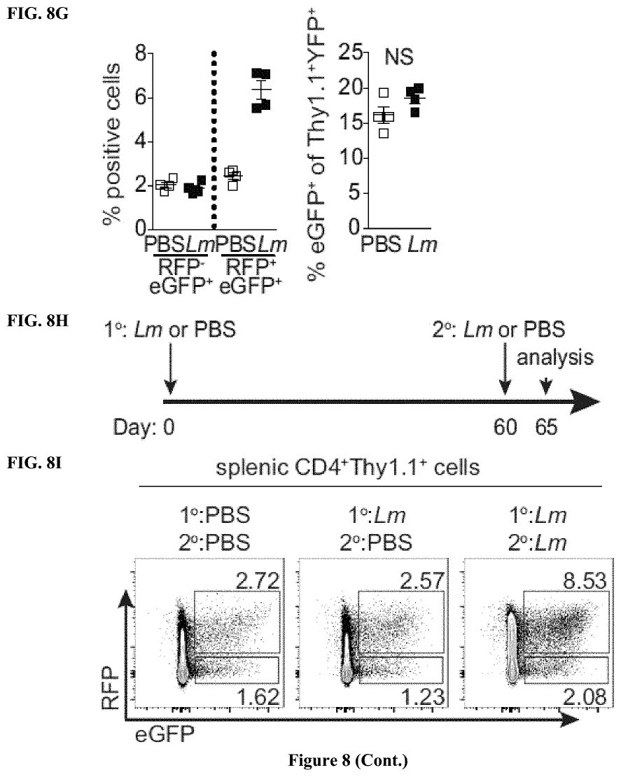

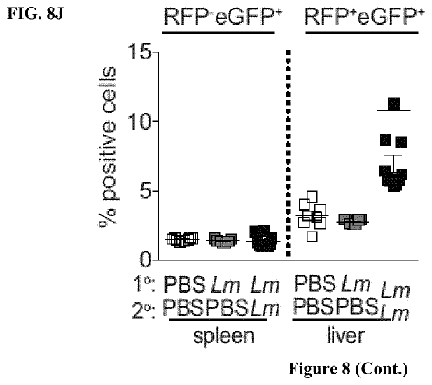

Three weeks after tamoxifen administration we found—in contrast to a previous report⁷—that the vast majority of both yellow fluorescent protein (YFP)-labelled Treg and effector CD4 T cells continued to express RFP (FIG. 1b, c, FIG. 6j). The percentage of YFP+ cells expressing RFP was similarly high at three and seven months after tamoxifen administration, although percentages of YFP+ cells declined, indicating that continual Treg cell recruitment into the T-bet+ subset balances out cell turnover over time (FIG. 1b, c, FIG. 6j). Indicative of intrinsic stability of T-bet+ Treg cells typical of a differentiated cell state, treatment of Tbx21RFP-creERT2 mice with tamoxifen 3 weeks before infection with the helminth Nippostrongylus brasiliensis did not result in loss of RFP expression among YFP+Treg (or effector CD4) T cells despite robust TH2 activation and cytokine production in the spleens and lungs of infected mice (FIG. 1d, FIG. 8a, data not shown).

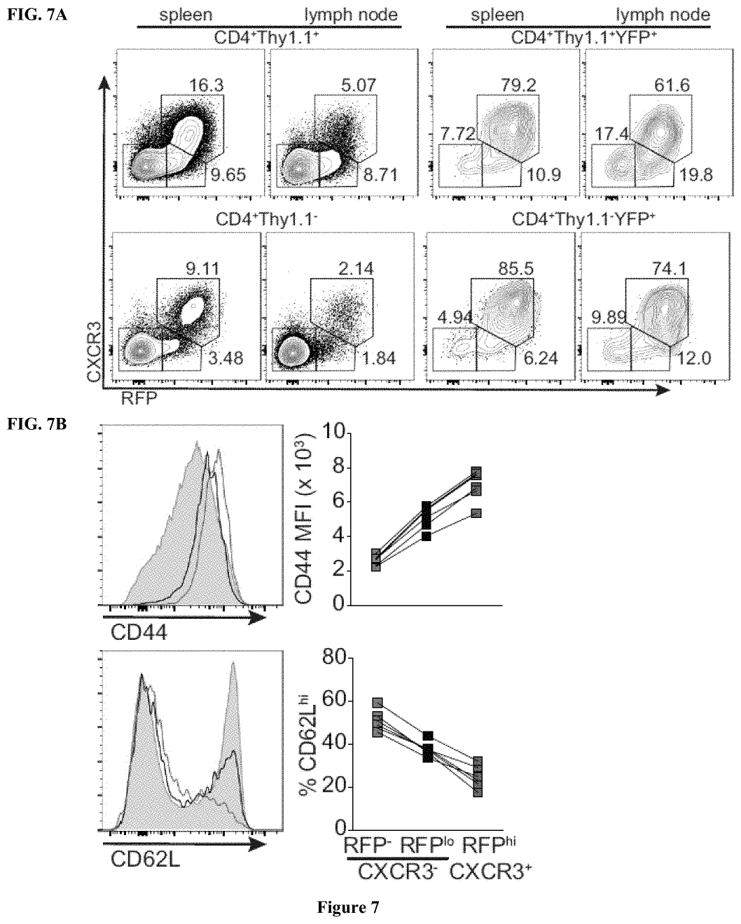

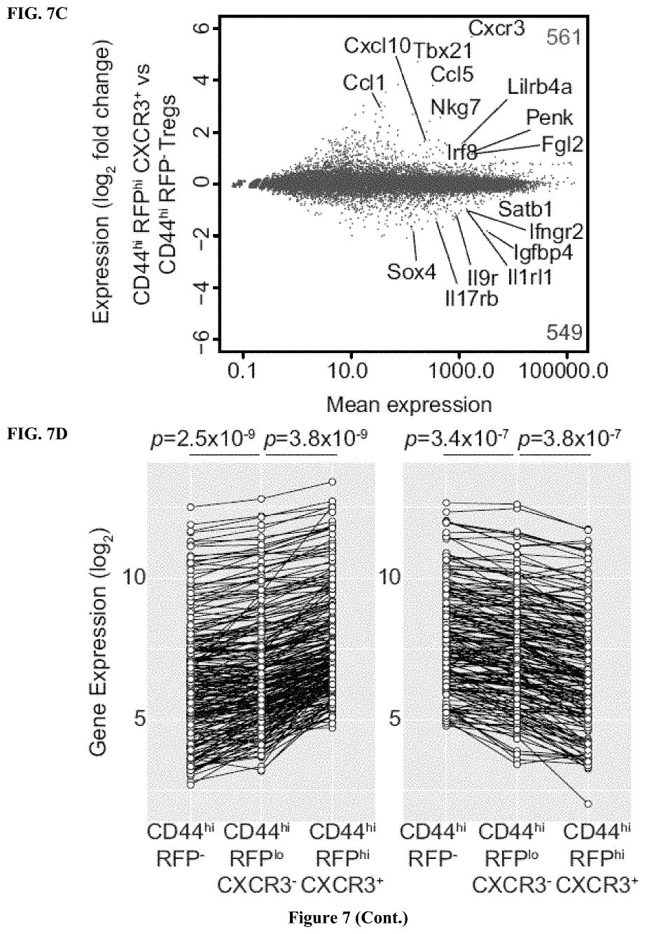

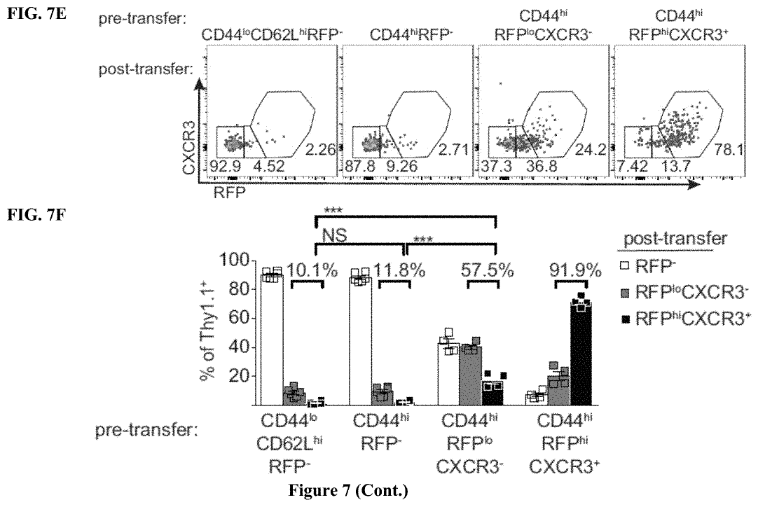

The presence of small percentages of YFP+RFP− cells 3 weeks after gavage (FIG. 1b, c) suggested that some Treg cells might have experienced transient unstable T-bet expression at the time of tamoxifen administration. Such a scenario would reconcile the above result with an earlier study⁷. Indeed, in Tbx21RFP-creERT2 mice we observed RFPlo Treg cells that lacked the T-bet-dependent expression of chemokine receptor CXCR3, in addition to RFPhiCXCR3+ cells (FIG. 7a). The former exhibited slightly lower CD44 and slightly higher CD62L expression than the latter and RNA sequencing (RNA-seq) analysis suggested that CD44hiRFPloCXCR3− Treg cells were differentiation intermediates between CD44hiRFP− cells and CD44hiRFPhiCXCR3+ cells (FIG. 7b-7d). Around 40% of FACS-sorted RFPloCXCR3− (but not RFPhiCXCR3+) Treg cells lost RFP expression following transfer into lymphoreplete hosts, whereas some became RFPhiCXCR3+(FIG. 7e, 7f). Notably, populations of RFPloCXCR3− and YFP+RFP− cells were also observed within the CD4 non-Treg cell population (FIG. 7a). Thus, the observed instability of a low level of T-bet expression is not unique to Treg cells but is indicative of the gradual process of peripheral T cell effector differentiation^{8,9}.

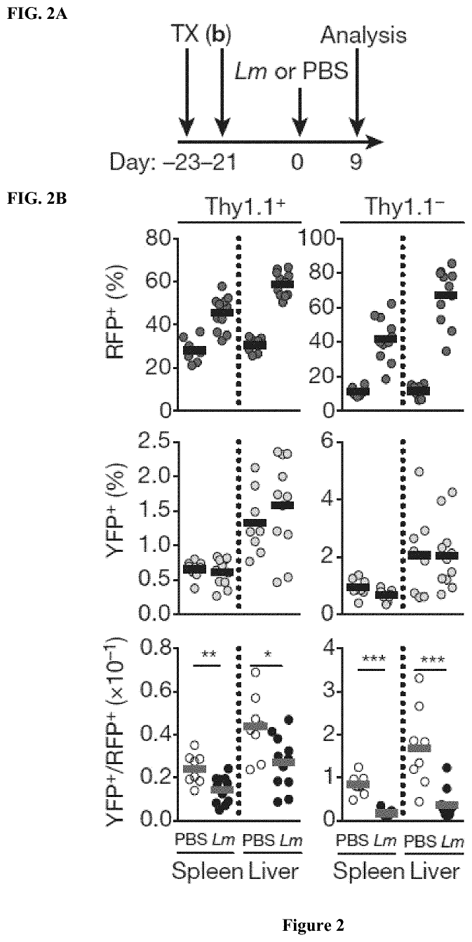

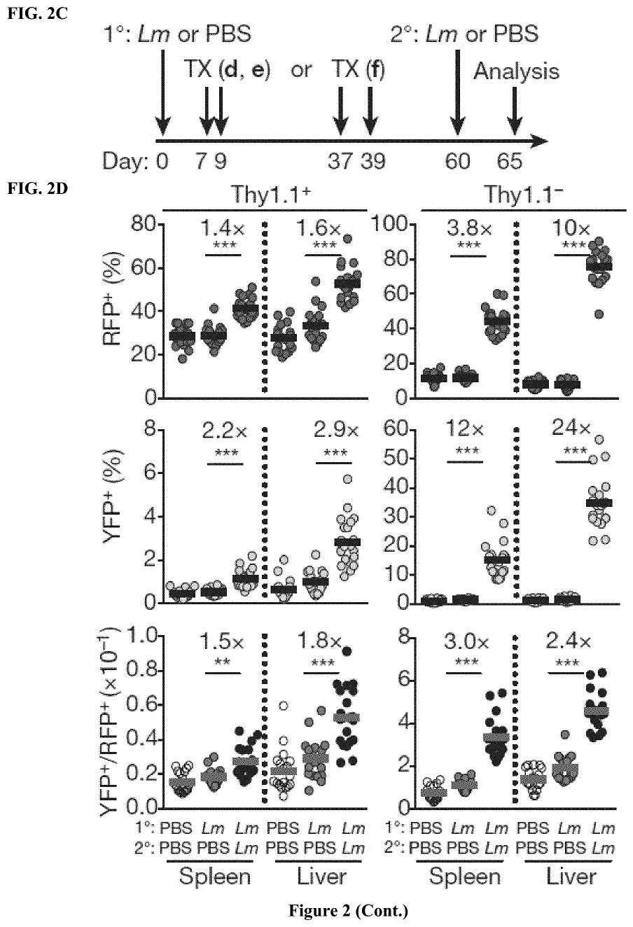

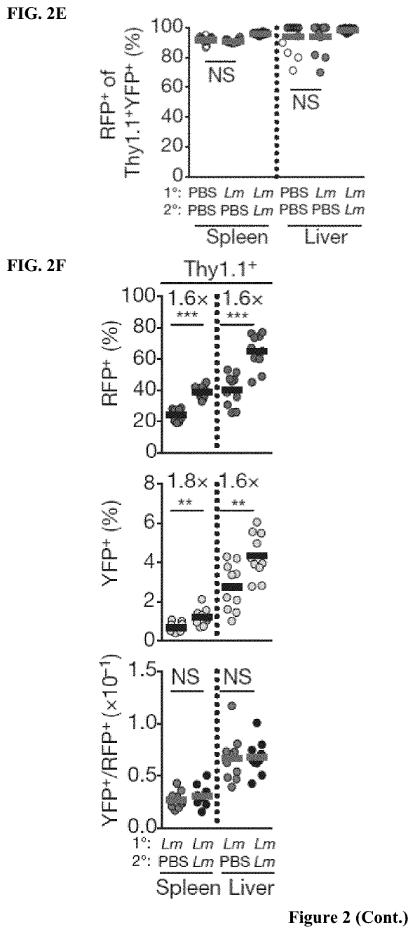

In addition to steady state cues, TH1-polarizing infection can drive increases in T-bet+ Treg cells¹⁰. To determine whether infection expands T-bet+ Treg cells present at steady state, or rather induces T-bet expression in T-bet− cells, we administered tamoxifen to Tbx21RFP-creERT2 mice 3 weeks before challenge with the intracellular bacteria Listeria monocytogenes. Upon L. monocytogenes challenge, RFP+ Treg and effector CD4 T cell subsets increased markedly; however, YFP+ subsets did not (yielding a decreased YFP+/RFP+ ratio) (FIG. 2a, b, FIG. 8b). This pattern was indicative of de novo differentiation of T-bet+ cells from T-bet− Treg precursors in parallel with differentiation of TH1 cells. Following transfer, both CD44loCD62Lhi RFP− and CD44hiRFP− Treg cells upregulated RFP in response to L. monocytogenes infection (FIG. 8c). Notably, upon L. monocytogenes infection, preformed T-bet+ Treg cells tagged with YFP prior to infection increased expression of T-bet and CXCR3, but not IL-10, an important suppressor molecule¹¹. The latter was demonstrated by fate mapping experiments in Tbx21RFP-creERT2Il10eGFP/WT mice, which revealed no increase in IL-10 (eGFP+) among YFP+ Treg cells, whereas bulk T-bet (RFP+) IL-10+ cells increased around threefold (FIG. 8d-8g). Similar results were obtained during lymphocytic choriomeningitis virus infection (data not shown).

We next assessed the persistence and recall response of T-bet+ Treg cells induced by L. monocytogenes infection. To preferentially label infection-induced T-bet+ cells, tamoxifen was administered at the peak of the primary L. monocytogenes response (days 7 and 9). Mice were assessed 8 weeks later, at which time the percentage of splenic and liver Treg cells that were RFP+ had returned to roughly pre-infection levels (FIG. 2c, d). Given the turnover rate of T-bet+ cells (FIG. 1c), we reasoned that by day 60 after infection YFP+ cells would be relatively enriched for infection-induced Treg cells compared to the bulk RFP+ cell pool. Reinfection increased bulk RFP+ Treg and effector CD4 T cells and even more prominently increased the corresponding cell subsets tagged with YFP (FIG. 2d, FIG. 8h-8j). On day 65 after primary infection, more than 90% of YFP+ Treg cells continued to express T-bet, as did uninfected control cells (FIG. 2e). Furthermore, mice infected with L. monocytogenes that were administered tamoxifen on days 37 and 39 after resolution of the primary response and re-infected on day 60 exhibited a parallel increase in bulk RFP+ and YFP+ Treg cell subsets on day 65, suggesting cells that acquired T-bet expression during primary infection remained T-bet-positive and expanded upon reinfection (FIG. 20. Together, these studies demonstrate that bacterial infection caused de novo differentiation of T-bet− Treg cells into stable T-bet+ cells uniquely suited for reactivation under conditions that drove their initial acquisition of T-bet.

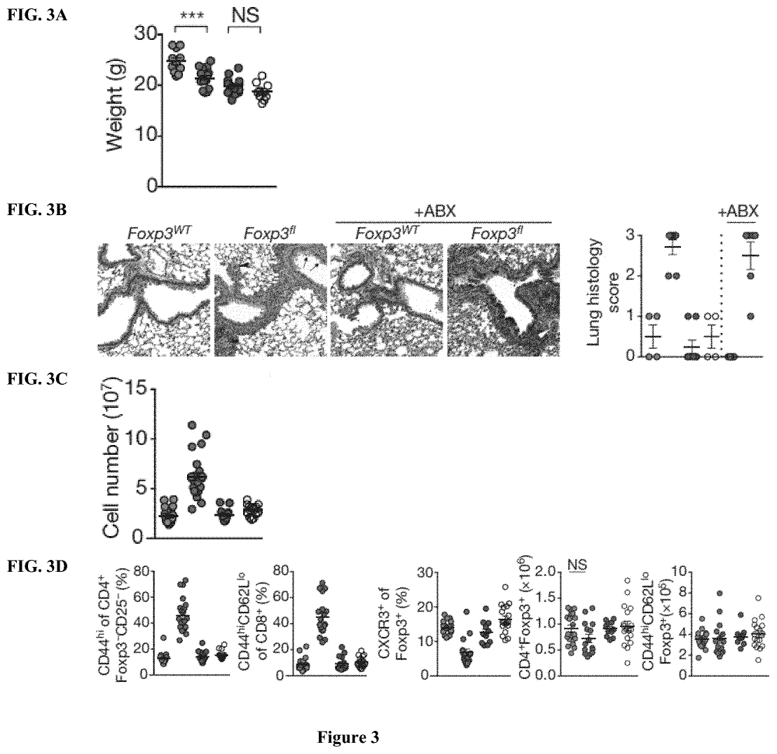

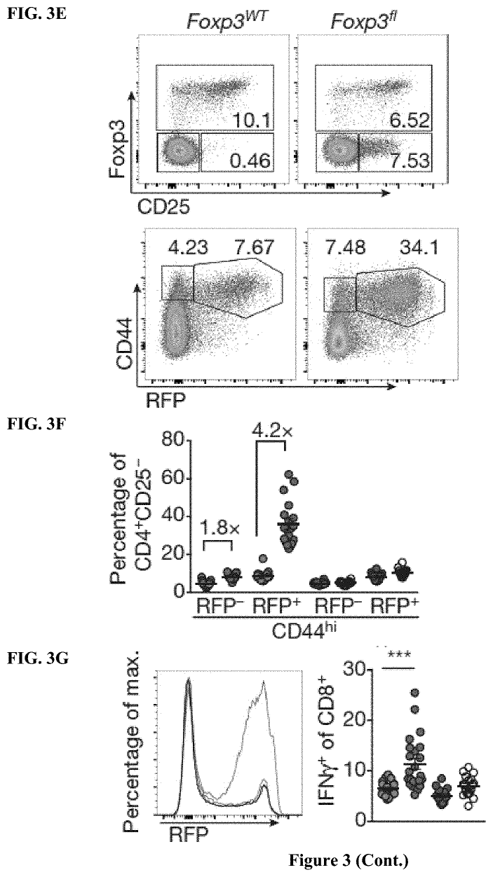

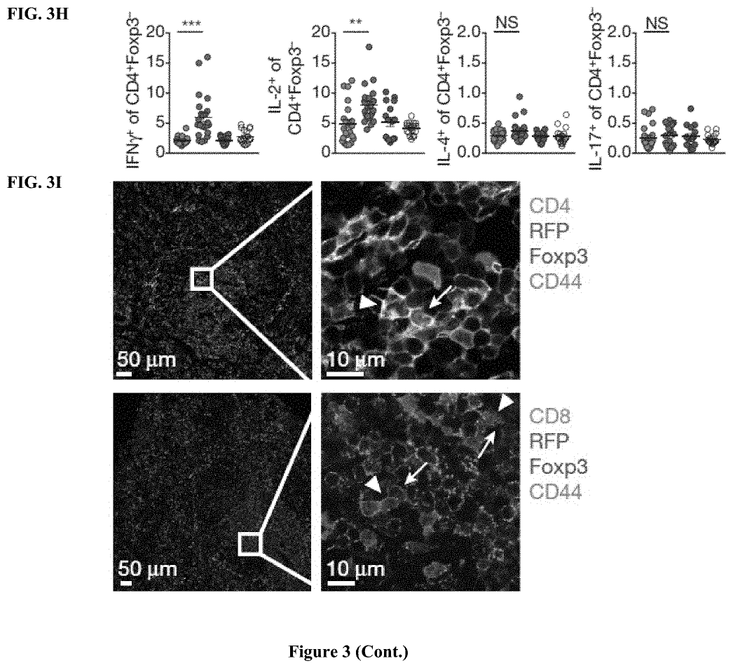

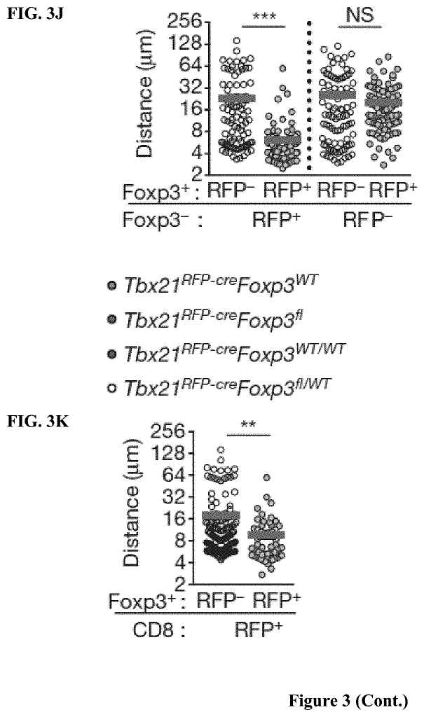

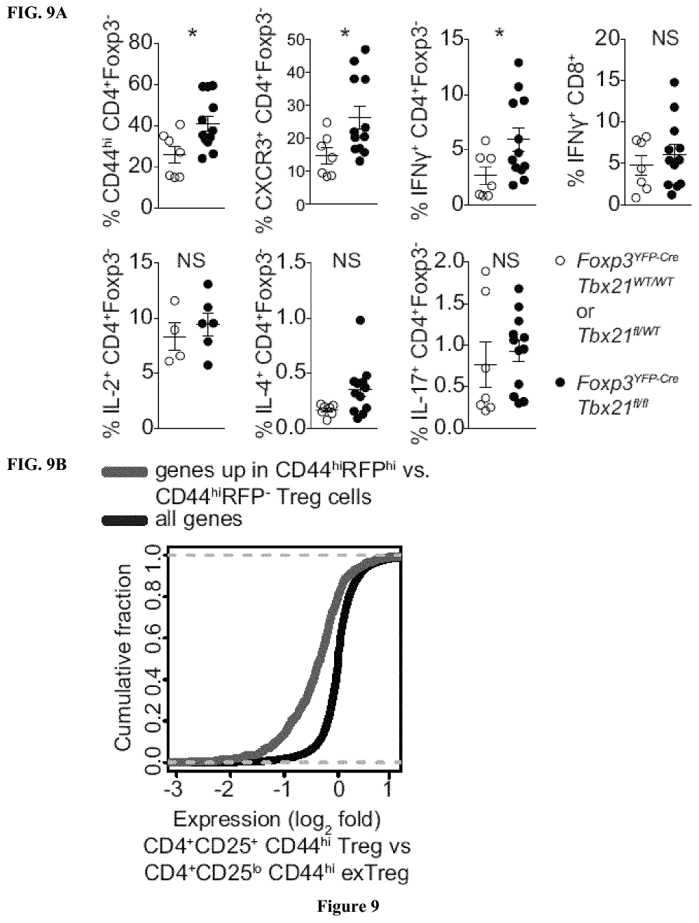

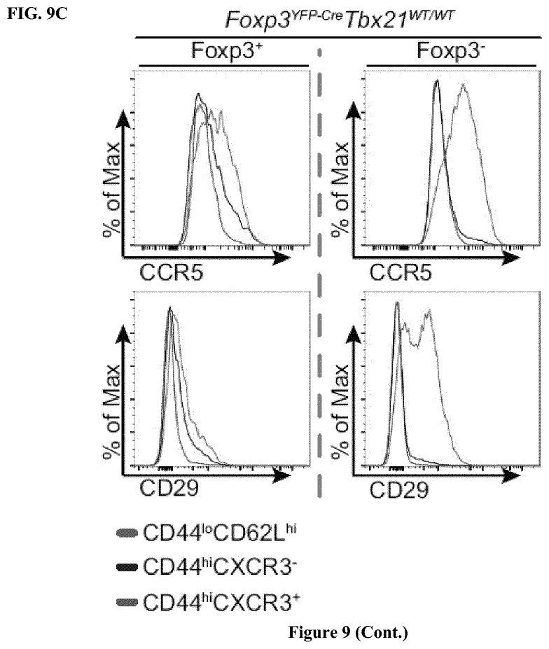

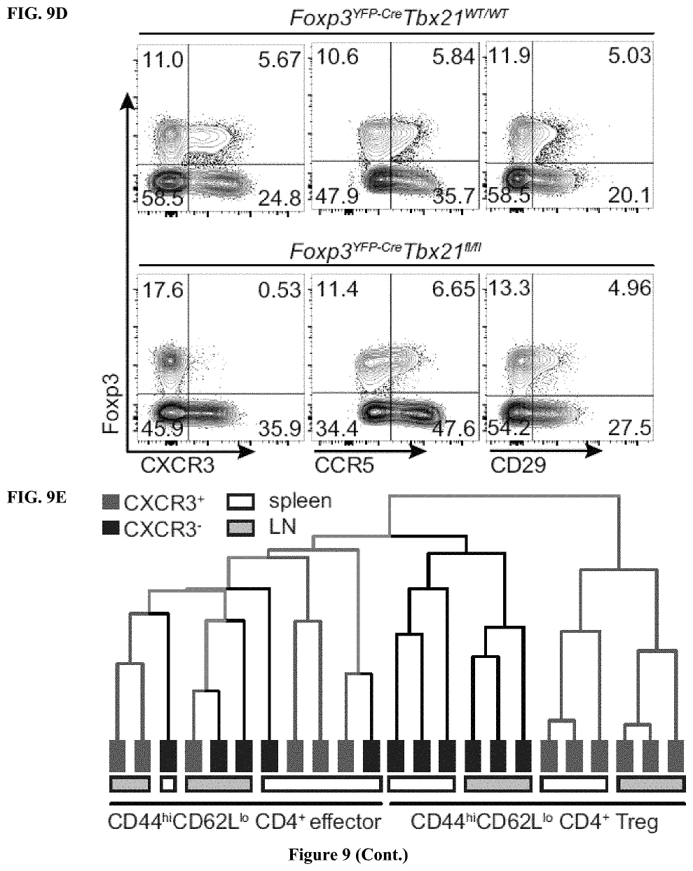

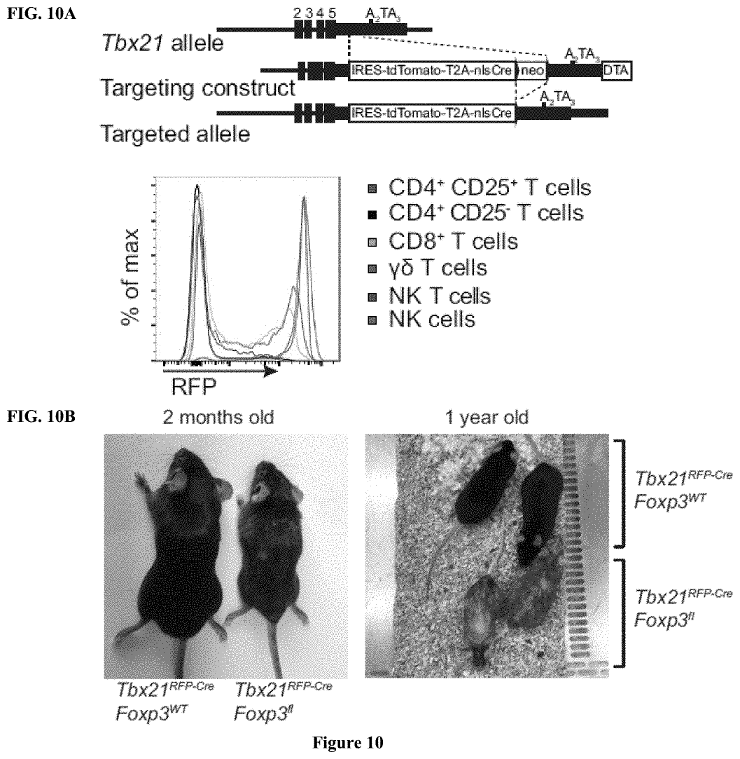

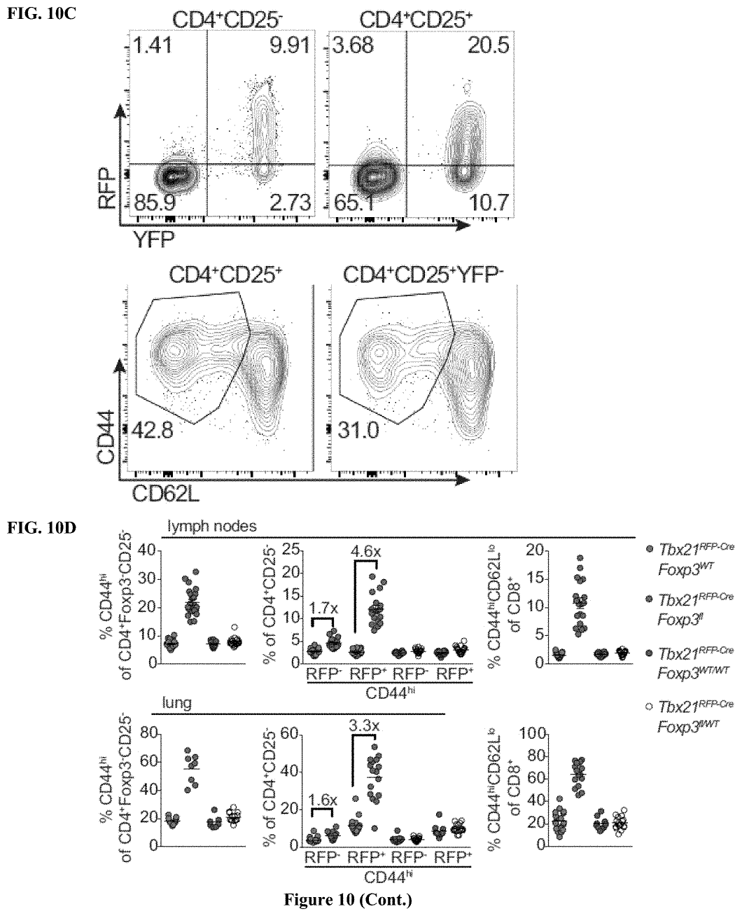

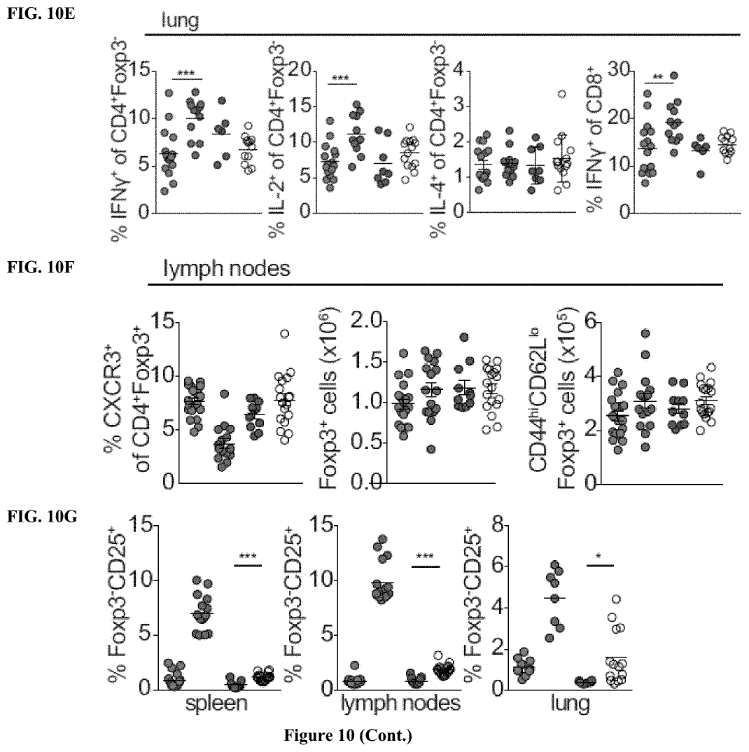

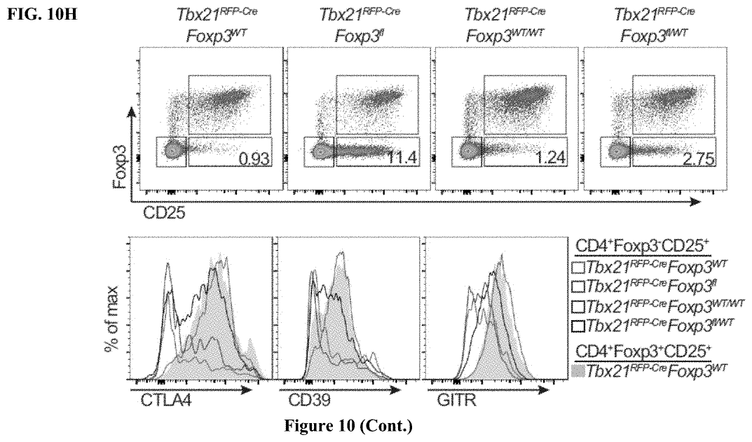

Although the stability of T-bet+ Treg cells suggested a particular function presumably imparted by T-bet itself, we found that 12-week-old Foxp3YFP-creTbx21fl/fl mice were clinically indistinguishable from littermate controls, consistent with previous studies7,12,13. Foxp3YFP-cre Tbx21fl/fl mice did exhibit mild TH1 (but not CD8 T cell) activation, indicating that T-bet expression in Treg cells moderately potentiated suppression of TH1 autoimmunity (FIG. 9a). We considered the possibility that T-bet deficiency might not fully impair the function of T-bet+ Treg cells. As Treg cell suppressor function requires continuous expression of the Foxp3 gene14, we ablated Foxp3 in T-bet+ Treg cells using a novel Tbx2ltdTomato-T2A-cre allele (FIG. 10a). Loss of Foxp3 expression in T-bet+ Treg cells in 8-week-old Tbx21RFP-creFoxp3fl mice resulted in deceased weight gain, lymphadenopathy, T cell activation, and marked immune infiltration in the lung; with age, loss of hair pigmentation and rectal prolapse were evident (FIG. 3a-d, FIG. 10).

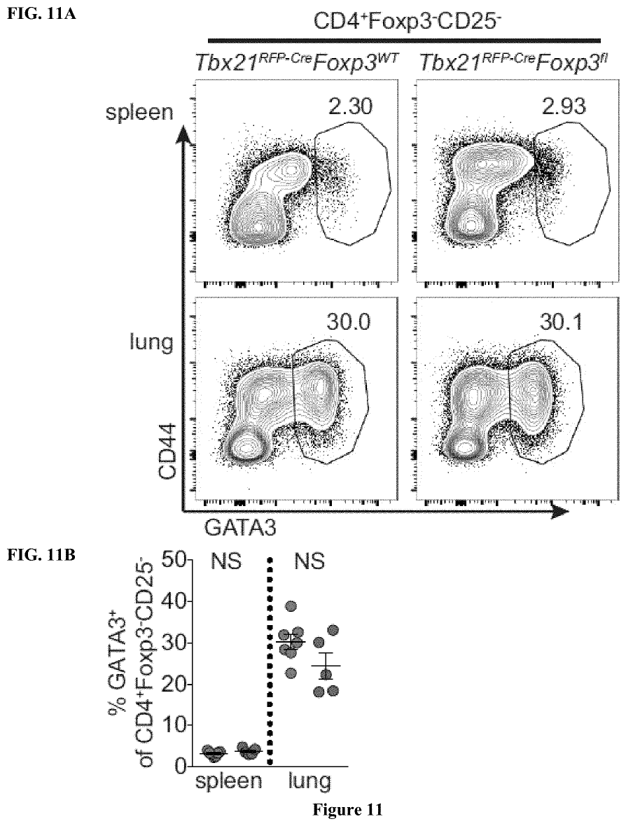

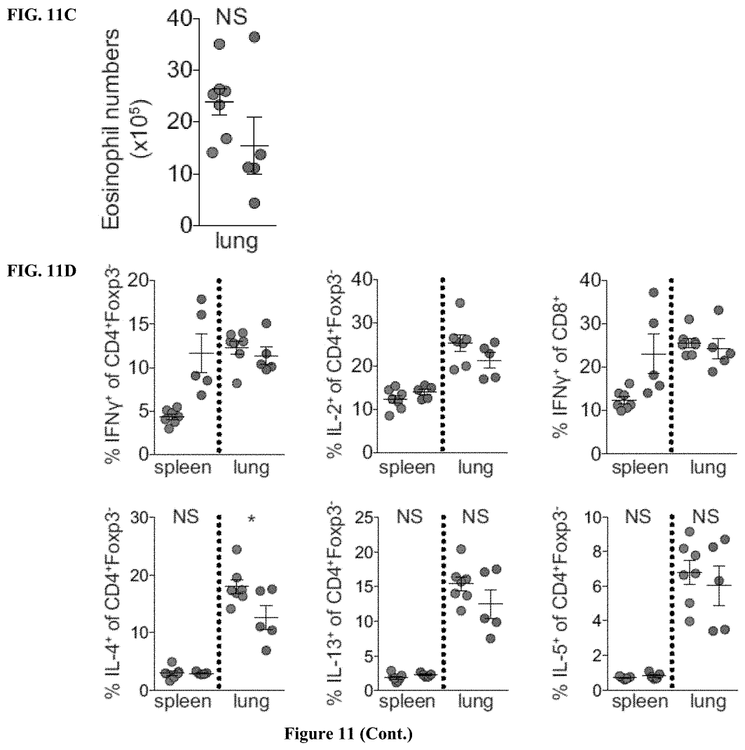

Indicative of TH1-type inflammation, the majority of expanded RFP (FIG. 3e-g, FIG. 10). Additionally, IFNγ and IL-2 but neither IL-4 nor IL-17 production by T cells were increased compared to controls (FIG. 3g, h, FIG. 10). Antibiotic treatment did not mitigate autoimmunity in Tbx21RFP-creFoxp3fl mice, excluding microbial antigens as the drivers of TH1 inflammation (FIG. 3b, data not shown). We considered whether induction of a robust non-TH1 immune response in Tbx21RFP-creFoxp3fl mice might reveal a potential function for T-bet+ Treg cells in its control. However, the TH2 response to N. brasiliensis infection was not increased in Tbx21RFP-creFoxp3fl mice compared to control mice, in contrast to the exacerbated TH2 response observed upon pan-Treg-cell depletion during helminth infection15,16 (FIG. 11). Notably, whereas CXCR3+ Treg cells were significantly depleted neither total nor effector Treg cell numbers were diminished, and analysis of Tbx21RFP-creR26Y mice confirmed that a significant proportion of effector Treg cells had not undergone Cre-mediated recombination (FIG. 3d, FIG. 10c, 100. These results suggested that immune activation could not be attributed to non-specific loss of effector Treg cells.

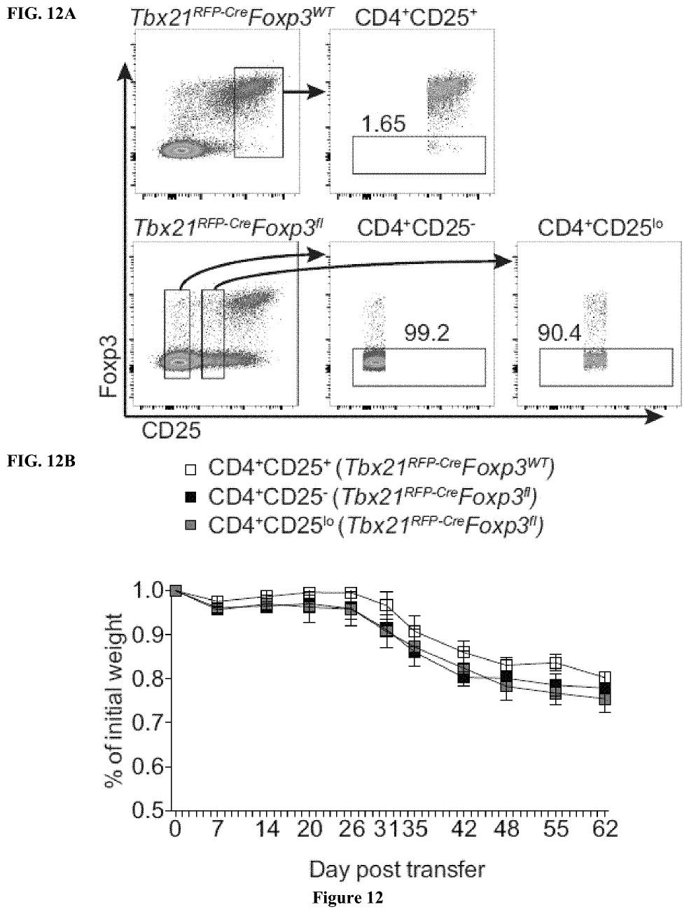

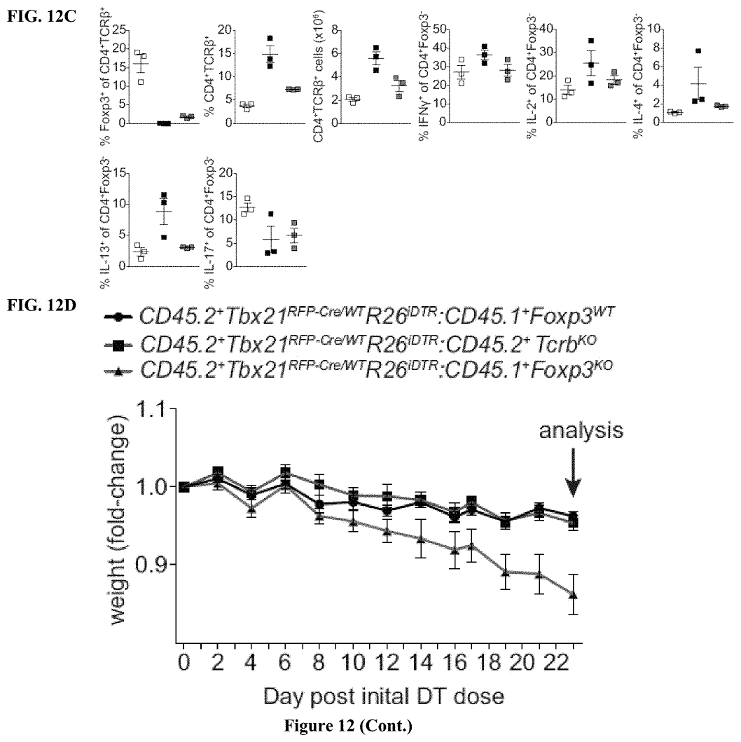

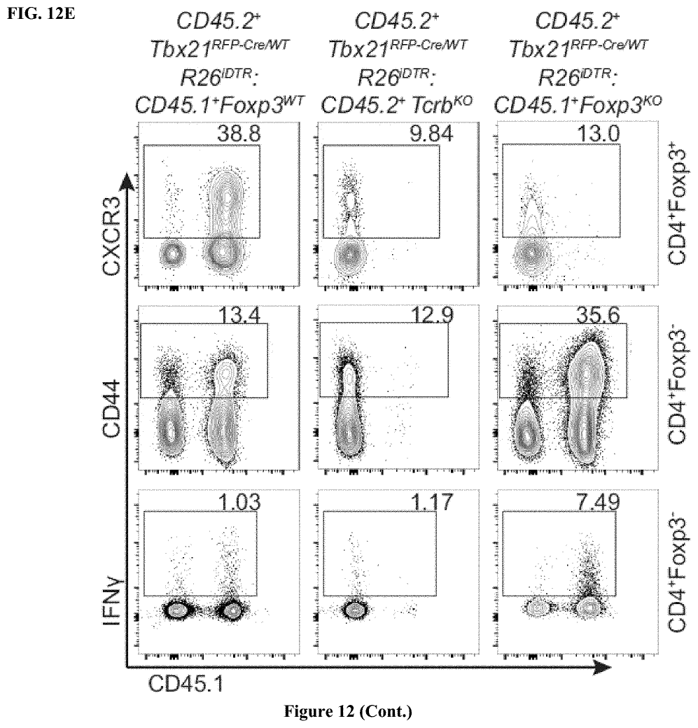

Cells that are likely to represent ex-Treg cells, which have lost Foxp3 expression, but continue to express high CD25, CD39, CTLA4 and GITR levels2,17,18 were readily found in male Tbx21RFP-creFoxp3fl and, to a lesser extent, female Tbx21RFP-creFoxp3fl/WT mice (FIG. 3e, FIG. 10g, 10h). The lack of autoimmunity in Tbx21RFP-creFoxp3fl/WT females—in which only half of T-bet+ Treg cells lose Foxp3 owing to X-inactivation—indicated that ex-Treg cells were efficiently controlled by remaining T-bet+ Treg cells (FIG. 3). Moreover, upon adoptive transfer into T-cell-deficient hosts, ex-Treg cells induced no more pathology and expanded less than CD4 effector T cells (FIG. 12a-c). These results indicate that ex-Treg cells were unlikely drivers of (although it is possible that they may play some role in) the observed autoimmunity.

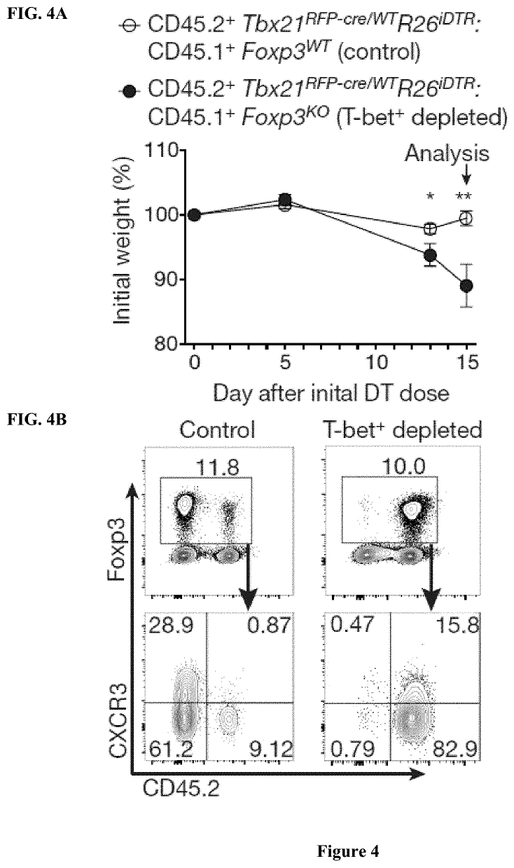

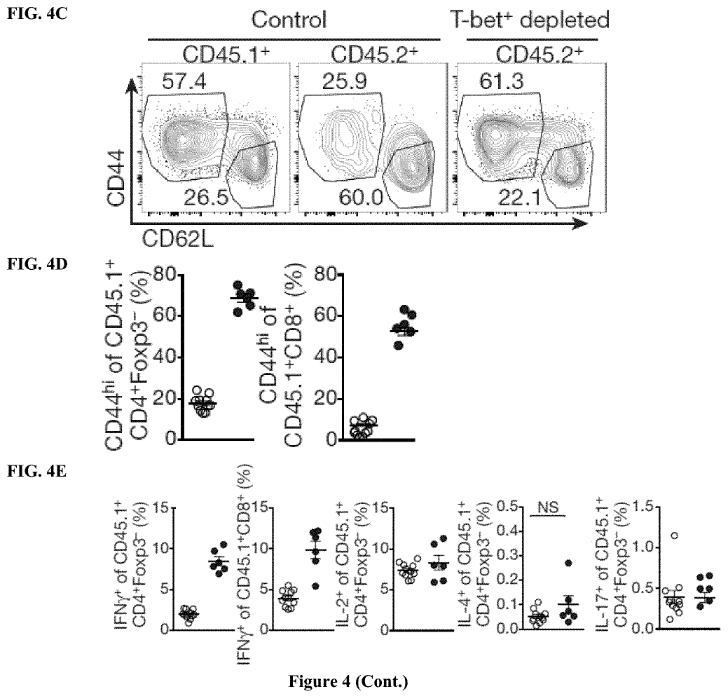

To determine whether punctual ablation of T-bet+ Treg cells would similarly unleash TH1 inflammation, we generated bone marrow chimaeric mice with a 1:1 mix of either CD45.1+Foxp3WT or Foxp3KO with CD45.2+Tbx21RFP-cre/WTR26iDTR haematopoietic precursor cells (FIG. 4). In Foxp3KO:Tbx21RFP-cre/WTRosa26iDTR mixed chimaeras, all T-bet+ Treg cells expressed diphtheria toxin receptor (DTR) and were susceptible to diphtheria-toxin-mediated ablation, whereas the rest of the T-bet-expressing cell types and subsets represented a 1:1 mix of DTR-expressing and non-expressing cells. Before treatment with diphtheria toxin, both sets of mixed chimaeras were healthy with similar basal levels of T cell activation (data not shown). Administration of diphtheria toxin over 2 weeks resulted in weight loss, T cell activation, and a selective increase in IFNγ production by CD4 and CD8 T cells in Foxp3KO:Tbx21RFP-cre/WTR26iDTR chimaeric mice ablated of T-bet+ Treg cells compared to Foxp3WT:Tbx21RFP-cre/WTR26iDTR controls (FIG. 4). Treg cell percentages in experimental mice were only very modestly decreased (from 13±0.53 to 11±0.82, P=0.022) and, as in Tbx21RFP-creFoxp3fl mice, percentages of CD44hiCD62Llo Treg cells were undiminished compared to controls (FIG. 4b, c). This experimental model is not confounded by generation of ex-Treg cells, providing additional evidence that the latter were not the sole drivers of pathology in the absence of T-bet+ Treg cells. Finally, weight loss was not observed in TcrbKO:Tbx21RFP-cre/WTR26iDTR mixed chimaeras, in which T-bet+ Treg and effector T-bet+ TCRαβ+ cells were simultaneously ablated, implicating the latter in driving disease (FIG. 12d, e).

RNA-seq analysis revealed that 561 genes, including Tbx21, Cxcr3, Gzmb, Ebi3, Fgl2, and Il10, were more highly expressed in CD44hiRFP+ cells compared to CD44hiRFP− Treg cells (FIG. 7c). Expression of this gene set was increased upon loss of Foxp3 in ex-Treg cells, suggesting that Foxp3 opposes the transcriptional signature of T-bet+ Treg cells to prevent full TH1 differentiation10 (FIG. 9b). Notably, the TH1-associated chemokine receptor CCR5 and adhesion molecule β1-integrin (CD29) were expressed in T-bet+ Treg cells independently of T-bet (FIG. 9c, d) indicating that some functional redundancy of homing molecules may in part explain the mild phenotype of Foxp3YFP-creTbx21fl/fl mice. Moreover, we found that the TCR repertoires of CD44hiCXCR3(T-bet)+ and CD44hiCXCR3(T-bet)− Treg subsets in DO11.10 TCRβ+Tcra+/− mice were distinct, suggesting that antigenic specificity of T-bet+ Treg cells may also contribute to distinct localization and suppressor capacity, as recent studies revealed TCR-dependent spatial proximity of Treg and IL-2-producing self-reactive T cells19 (FIG. 9e).

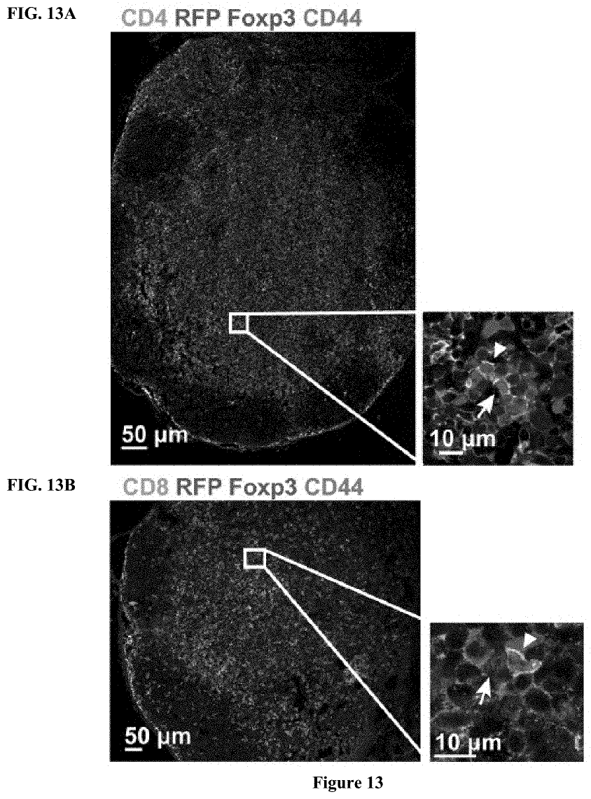

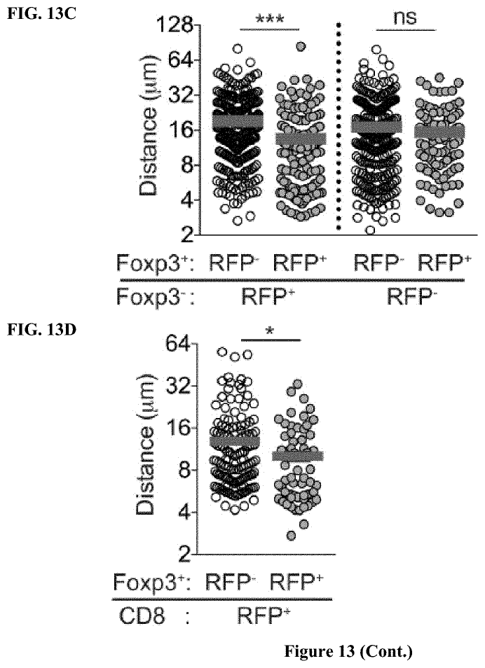

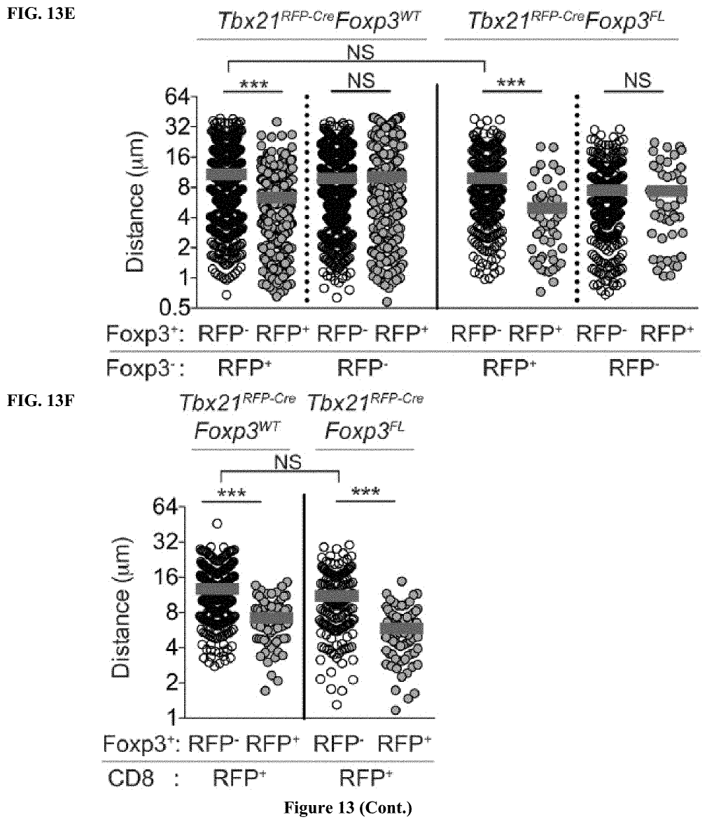

Therefore, we sought to determine the relative spatial positioning of T-bet+ and T-bet− Treg and effector T cells in secondary lymphoid organs of Tbx21RFP-cre mice. Immunofluorescence imaging revealed pronounced preferential proximity of CD44hiT-bet+ versus CD44hiT-bet− Treg cells to CD44hiT-bet+TH1 and CD8 T cells (FIG. 3i-k, FIG. 13a-13d). In contrast, CD44hiT-bet+ Treg cells were no nearer to T-bet−CD4 effectors than were CD44hiT-bet− Treg cells (FIG. 3j, FIG. 13c). Notably, the CD44hiT-bet− Treg cells remaining in Tbx21RFP-creFoxp3fl mice were no nearer to TH1 or CD8 T cells than were CD44hiRFP− Treg cells in healthy Tbx21RFP-creFoxp3WT mice (FIG. 13e, f). This result suggests that failure of non-T-bet+ Treg cells to approximate TH1 cells may at least in part account for their inability to suppress TH1 inflammation.

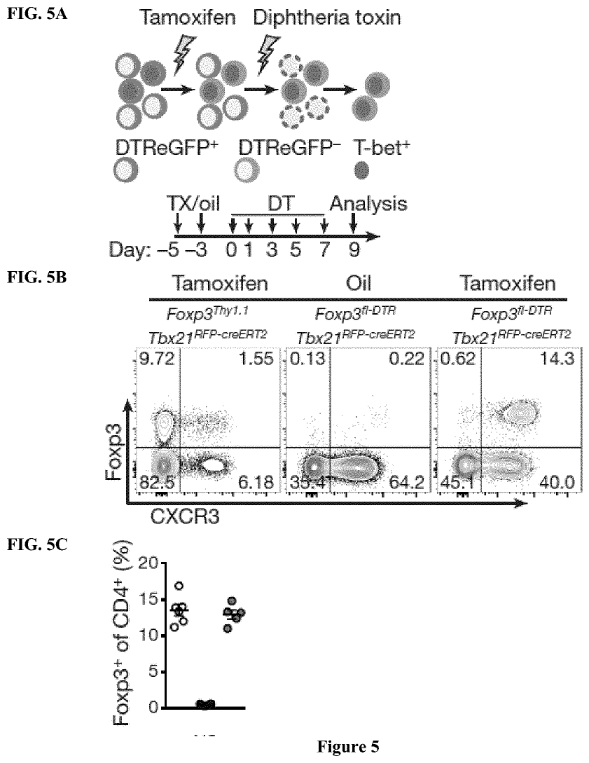

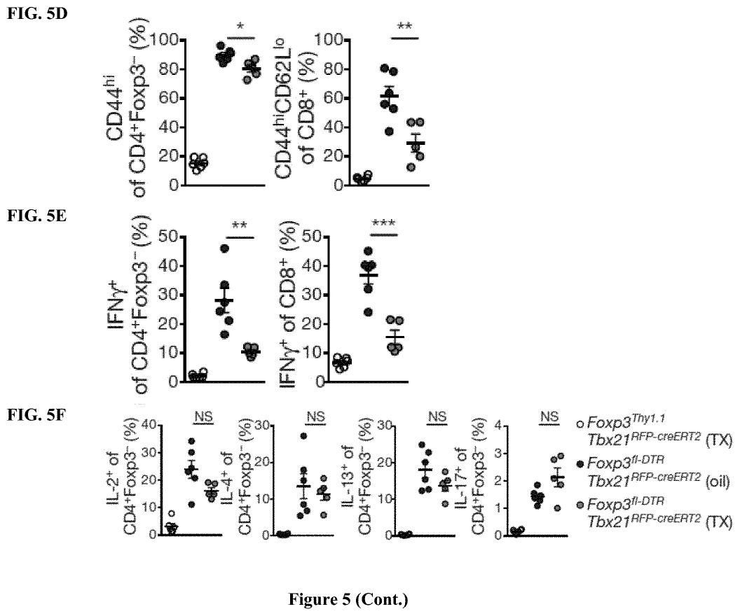

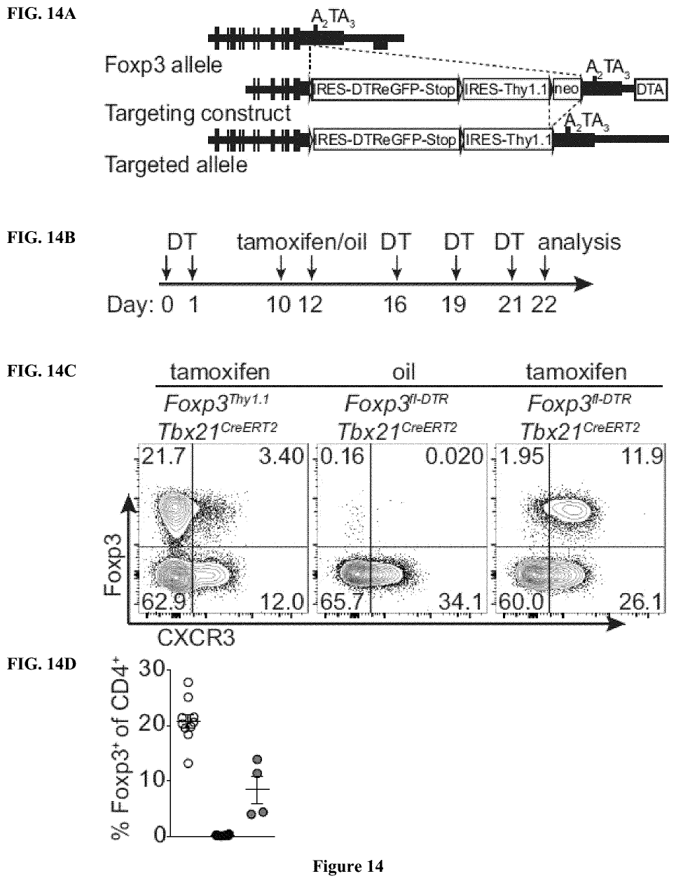

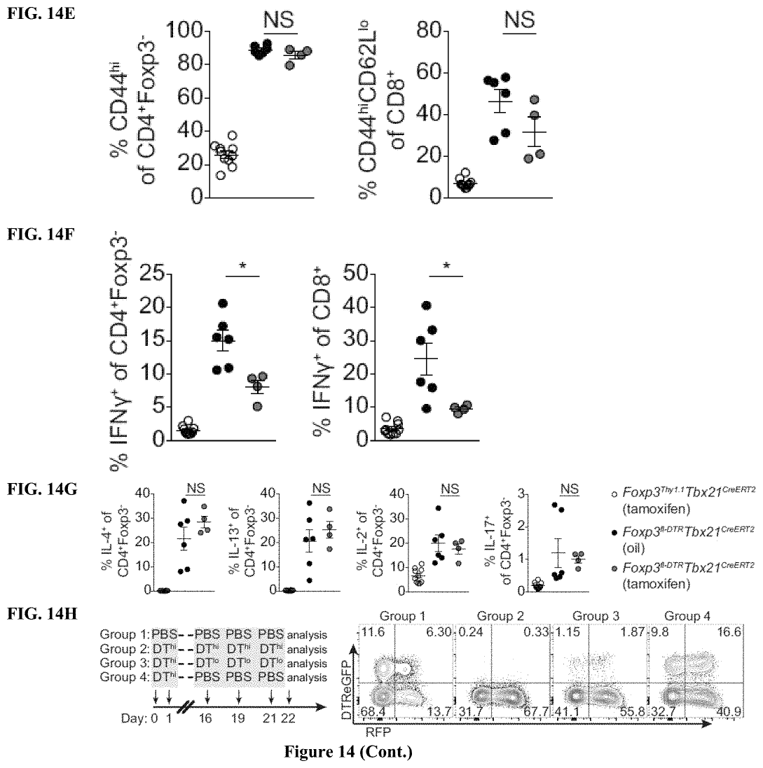

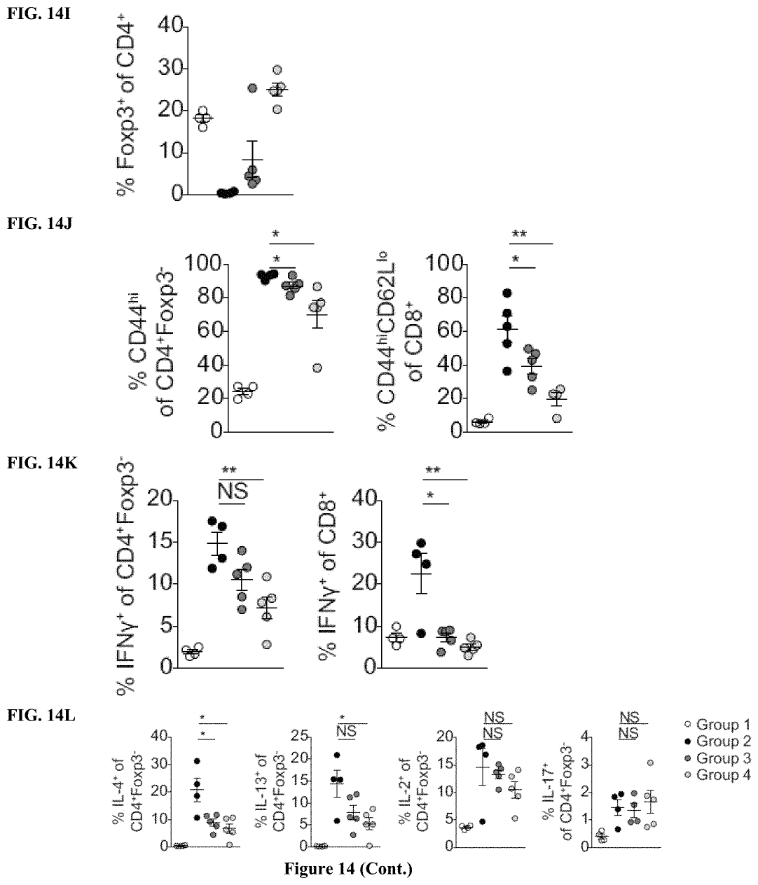

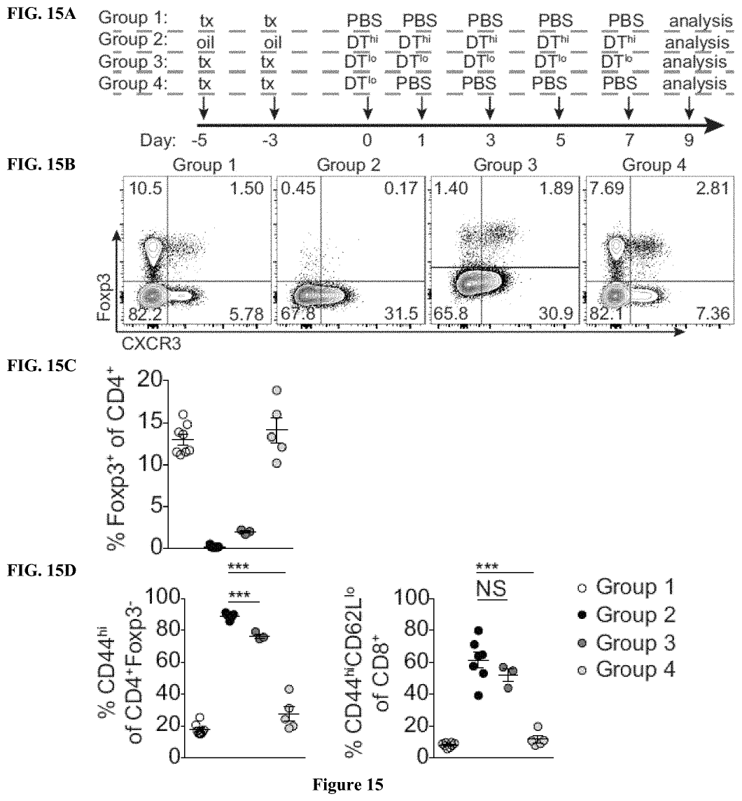

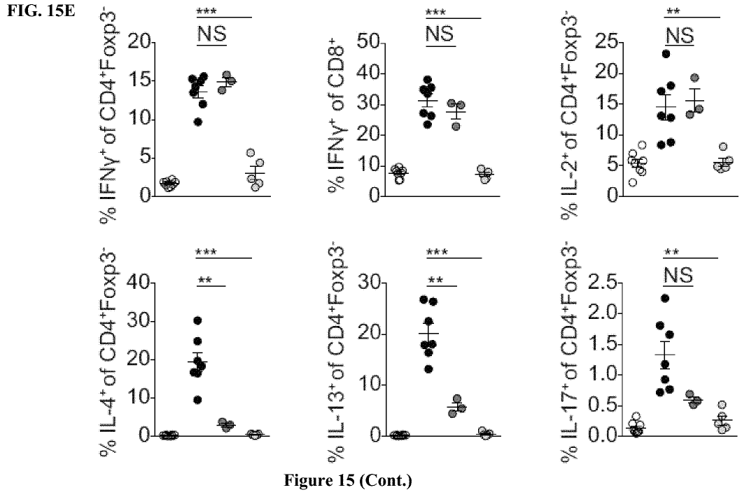

Lastly, to complement T-bet+ Treg cell ‘loss-of-function’ experiments we sought to selectively eliminate T-bet− Treg cells. We generated a Foxp3fl-DTR allele by inserting a loxP-flanked IRES-DTReGFP DNA sequence into the 3′ UTR of the Foxp3 gene (FIG. 14a) and generated Foxp3fl-DTRTbx21RFP-creERT2 mice (FIG. 5a). After 9 days of diphtheria toxin treatment, Treg cells in mice pre-treated with tamoxifen (day −5 and −3) were present in undiminished percentages and were exclusively T-bet+ and CXCR3+(FIG. 5a-c). Compared to vehicle (oil)-treated mice, tamoxifen-treated Foxp3fl-DTRTbx21RFP-creERT2 mice displayed robustly suppressed CD8 T cell activation and selective suppression of IFNγ production by CD4 and CD8 T cells, but unrestrained TH2 and TH17 cytokine production (FIG. 5d-f). T-bet+ Treg cells similarly suppressed pre-established TH1, but not TH2 or TH17, activation induced by depletion of Treg cells before tamoxifen treatment (FIG. 14b-14g). Selective TH1 suppression was not simply a feature of activated Treg cells rebounding after depletion, as partial depletion and recovery of Treg cells in Foxp3DTR mice resulted in prominently inhibited TH2 responses (FIGS. 14h-14l, 15).

Our studies suggest that T-bet expression in Treg cells denotes a differentiated cell state with unique T-bet-dependent and—independent gene expression and TCR specificity, capable of driving potent immunosuppression limited to circumstances of TH1 and CD8 T cell activation. Such division of anti-inflammatory labour among Treg cells, arising at steady state and during infection, may enable focused regulation of specific T helper cell responses without incurring undesired bystander suppression.

### REFERENCES

1. Zhu, J., Yamane, H. & Paul, W. E. Differentiation of effector CD4 T cell populations. Annu. Rev. Immunol. 28, 445-489 (2010).
2. Fontenot, J. D., Gavin, M. A. & Rudensky, A. Y. Foxp3 programs the development and function of CD4+CD25+ regulatory T cells. Nat. Immunol. 4, 330-336(2003).
3. Hori, S., Nomura, T. & Sakaguchi, S. Control of regulatory T cell development by the transcription factor Foxp3. Science 299, 1057-1061 (2003).
4. Koch, M. A. et al. The transcription factor T-bet controls regulatory T cell homeostasis and function during type 1 inflammation. Nat. Immunol. 10, 595-602 (2009).
5. Ohnmacht, C. et al. Mucosal immunology. The microbiota regulates type 2 immunity through RORγ t+ T cells. Science 349, 989-993 (2015).
6. Sefik, E. et al. Mucosal immunity. Individual intestinal symbionts induce a distinct population of RORγ+ regulatory T cells. Science 349, 993-997 (2015).
7. Yu, F., Sharma, S., Edwards, J., Feigenbaum, L. & Zhu, J. Dynamic expression of transcription factors T-bet and GATA-3 by regulatory T cells maintains immunotolerance. Nat. Immunol. 16, 197-206 (2015).
8. Zhou, L. et al. TGF-β-induced Foxp3 inhibits TH17 cell differentiation by antagonizing RORγ t function. Nature 453, 236-240 (2008).
9. Hwang, E. S., Szabo, S. J., Schwartzberg, P. L. & Glimcher, L. H. T helper cell fate specified by kinase-mediated interaction of T-bet with GATA-3. Science 307, 430-433 (2005).
10. Koch, M. A. et al. T-bet+ Treg cells undergo abortive TH1 cell differentiation due to impaired expression of IL-12 receptor β2. Immunity 37, 501-510 (2012).
11. Laidlaw, B. J. et al. Production of IL-10 by CD4+ regulatory T cells during the resolution of infection promotes the maturation of memory CD8+ T cells. Nat. Immunol. 16, 871-879 (2015).
12. Colbeck, E. J. et al. Eliminating roles for T-bet and IL-2 but revealing superior activation and proliferation as mechanisms underpinning dominance of regulatory T cells in tumors. Oncotarget 6, 24649-24659 (2015).
13. McPherson, R. C., Turner, D. G., Mair, I., O'Connor, R. A. & Anderton, S. M. T-bet expression by Foxp3+T regulatory cells is not essential for their suppressive function in CNS autoimmune disease or colitis. Front. Immunol. 6, 69 (2015).
14. Williams, L. M. & Rudensky, A. Y. Maintenance of the Foxp3-dependent developmental program in mature regulatory T cells requires continued expression of Foxp3. Nat. Immunol. 8, 277-284 (2007).
15. Smith, K. A. et al. Low-level regulatory T-cell activity is essential for functional type-2 effector immunity to expel gastrointestinal helminths. Mucosal Immunol. 9, 428-443 (2016).
16. Sawant, D. V. et al. Regulatory T cells limit induction of protective immunity and promote immune pathology following intestinal helminth infection. J. Immunol. 192, 2904-2912 (2014).
17. Lahl, K. et al. Nonfunctional regulatory T cells and defective control of TH2 cytokine production in natural scurfy mutant mice. J. Immunol. 183, 5662-5672 (2009).
18. Lin, W. et al. Regulatory T cell development in the absence of functional Foxp3. Nat. Immunol. 8, 359-368 (2007).
19. Liu, Z. et al. Immune homeostasis enforced by co-localized effector and regulatory T cells. Nature 528, 225-230 (2015).
20. Intlekofer, A. M. et al. Anomalous type 17 response to viral infection by CD8+ T cells lacking T-bet and eomesodermin. Science 321, 408-411 (2008).
21. Liston, A. et al. Differentiation of regulatory Foxp3+ T cells in the thymic cortex. Proc. Natl Acad. Sci. USA 105, 11903-11908 (2008).
22. Rubtsov, Y. P. et al. Regulatory T cell-derived interleukin-10 limits inflammation at environmental interfaces. Immunity 28, 546-558 (2008).
23. Srinivas, S. et al. Cre reporter strains produced by targeted insertion of EYFP and ECFP into the ROSA26 locus. BMC Dev. Biol. 1, 4 (2001).
24. Eberl, G. et al. An essential function for the nuclear receptor RORγ tin the generation of fetal lymphoid tissue inducer cells. Nat. Immunol. 5, 64-73 (2004).
25. Kamanaka, M. et al. Expression of interleukin-10 in intestinal lymphocytes detected by an interleukin-10 reporter knockin tiger mouse. Immunity 25, 941-952 (2006).
26. Buch, T. et al. A Cre-inducible diphtheria toxin receptor mediates cell lineage ablation after toxin administration. Nat. Methods 2, 419-426 (2005).
27. Camberis, M., Le Gros, G. & Urban, J., Jr. Animal model of Nippostrongylus brasiliensis and Heligmosomoides polygyrus. Curr. Prot. Immunol. 19, 19.12 (2003).
28. Li, H. & Durbin, R. Fast and accurate long-read alignment with Burrows-Wheeler transform. Bioinformatics 26, 589-595 (2010).
29. McKenna, A. et al. The Genome Analysis Toolkit: a MapReduce framework for analyzing next-generation DNA sequencing data. Genome Res. 20, 1297-1303 (2010).
30. Love, M. I., Huber, W. & Anders, S. Moderated estimation of fold change and dispersion for RNA-seq data with DESeq2. Genome Biol. 15, 550 (2014).
31. Feng, Y. et al. A mechanism for expansion of regulatory T-cell repertoire and its role in self-tolerance. Nature 528, 132-136 (2015).

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

We claim:

1. An isolated population of regulatory T (Treg) cells which have been engineered to express Tbet.

2. The population of claim 1, characterized by 2, 5, 10, 20, 30, 40, 50, 60 fold greater expression of Tbet relative to a reference.

3. The population of claim 1, characterized by an ability to suppress an immune response when contacted with a system undergoing or at risk of the immune response.

4. The population of claim 3, wherein the immune response is a Th1 type immune response.

5. The population of claim 4, wherein suppression of a T_{H}1 type immune response comprises reduced expression of IFNγ or IL-2 in the system.

6. The population of claim 1, wherein 90%, 80%, 70%, 60%, 50%, 40%, 30%, 20% of the Treg population expresses Tbet.

7. A method of suppressing a T_{H}1 type immune response the method comprising administering to a subject a population of Tregs which have been engineered to express Tbet.

8. The method of claim 7, further comprising obtaining Tregs.

9. The method of claim 8, wherein the Tregs are obtained from the subject.

10. The method of claim 8, wherein the Tregs are obtained from an individual other than the subject

11. The method of claim 7, wherein engineering comprises increasing expression of Tbet in the obtained Tregs.

12. The method of claim 11, wherein increased expression of Tbet comprises stimulation of the obtained Tregs in the presence of IFNγ and IL-27.

13. The method of claim 11, wherein engineering comprises introducing a vector expressing Tbet in to the obtained Tregs.

14. The method of claim 13, wherein the vector is a viral vector.

15. The method of claim 7, wherein engineering comprises increasing expression of Tbet through transfection of the obtained Tregs with a nucleic acid or amino acid sequence encoding Tbet.

16. A method of preparing an specialized Treg population, the method comprising:
obtaining an initial Treg cell or population
culturing the initial Treg cell or population for a period of time and under conditions sufficient that a specialized Treg population characterized in that Tbet expression is increased 2, 5, 10, 20, 30, 40, 50, 60 fold relative to a reference is prepared.

17. The method of claim 16, wherein the initial Treg cell or population has been engineered to express Tbet.

18. The method of claim 17, wherein the engineering comprises stimulation of the obtained Tregs in the presence of IFNγ and IL-27.

19. The method of claim 17, wherein the engineering comprises introducing a vector expressing Tbet in to the obtained Tregs.

20. The method of claim 19, wherein the vector is a viral vector.

21. The method of claim 17, wherein the engineering comprises transfection of the obtained Tregs with a nucleic acid or amino acid sequence encoding Tbet.

22. The method of claim 16, wherein the initial Treg cell or population is isolated from a subject.

23. The method of claim 22, wherein the subject is a human subject.

24. The method of claim 23, wherein the subject is suffering from or susceptible to a disease disorder or condition characterized by inflammation or autoimmunity.

25. The method of claim 24, wherein the subject is suffering from or susceptible to a disease disorder or condition characterized by a Th1 immune response.

26. The method of claim 16 further comprising steps of:
isolating the specialized Treg population; or
combining the specialized Treg population with a pharmaceutically acceptable carrier or excipient so that a pharmaceutical composition comprising the specialized Treg population is manufactured.
